SEQUENCE LISTING (17 sequences)

SEQ ID NO: 1 - DNA, 56 bp, Artificial
  primer_bind
ctagacagccctgatgagtccgtgaggacgaaacttgatcaaagtcggctgtaggc

SEQ ID NO: 2 - DNA, 60 bp, Artificial
  primer_bind
caatttatgcctacagccgactttgatcaagtttcgtcctcacggactcatcagggctgt

SEQ ID NO: 3 - DNA, 60 bp, Artificial
  primer_bind
ataaattggtttgcgaggtgcgcactgatgagtccgtgaggacgaaaccaattactagtg

SEQ ID NO: 4 - DNA, 56 bp, Artificial
  primer_bind
aattcactagtaattggtttcgtcctcacggactcatcagtgcgcacctcgcaaac

SEQ ID NO: 5 - DNA, 56 bp, Artificial
  primer_bind
ctagaattggctgatgagtccgtgaggacgaaacttgatcaaagtcccaatttatg

SEQ ID NO: 6 - DNA, 60 bp, Artificial
  primer_bind
gctgtaggcataaattgggactttgatcaagtttcgtcctcacggactcatcagccaatt

SEQ ID NO: 7 - DNA, 59 bp, Artificial
  primer_bind
cctacagccttagtaggtgacctctgatgagtccgtgaggacgaaaggctgtactagtg

SEQ ID NO: 8 - DNA, 56 bp, Artificial
  primer_bind
aattcactagtacagcctttcgtcctcacggactcatcagaggtcacctacctaag

SEQ ID NO: 9 - DNA, 207 bp, Artificial
  misc_signal
gatcctcgagtctagacgcctgatgagtccgtgaggacgaaacgaattctgatcagaattcgtcgcgcgaccacggggcgcacctctctttactgtgaagccacagatgggcaaagagagatgcgccccatggccgtgctcgggcccccgcgggggcccctgatgagaggagtgcctcgaaagcaactagtgcggccgctaaactat

SEQ ID NO: 10 - DNA, 207 bp, Artificial
  misc_signal
gatcctcgagtctagacgcctgatgagtccgtgaggacgaaacgaattctgatcagaattcgtcgcgccgtgtgcacttcgcttcacctctgctgtgaagccacagatgggcagaggcgaagcaaagcgcacacgatgctcgggcccccgcgggggcccctgatgagaggagtgcctcgaaagcaactagtgcggccgctaaactat

SEQ ID NO: 11 - DNA, 207 bp, Artificial
  misc_signal
gatcctcgagtctagacgcctgatgagtccgtgaggacgaaacgaattctgatcagaattcgtcgcgttcaagcctccaagctgtgccttggctgtgaagccacagatgggccaaagcacaactcggaggctcgaatgctcgggcccccgcgggggcccctgatgagaggagtgcctcgaaagcaactagtgcggccgctaaactat

SEQ ID NO: 12 - DNA, 231 bp, Artificial
  misc_signal
ctagacagccctgatgagtccgtgaggacgaaacttgatcaaagtcggctgtaggcataaattggtttgcgaggtgcgcactgatgagtccgtgaggacgaaaccaattactagtgctagaattggctgatgagtccgtgaggacgaaacttgatcaaagtcccaatttatgcctacagccttagtaggtgacctctgatgagtccgtgaggacgaaaggctgtactagtg

SEQ ID NO: 13 - DNA, 3221 bp, Hepatitis B virus
ttccacagctttccaccaagctctgcaagatcccagagtcaggggcctgtattttcctgctggtggctccagttcaggaacactcaaccctgttccaaatattgcatctcacatctcgtcaatctcctcgaggattggggaccctgcgctgaacatggagaacattacatcaggattcctaggacccctgctcgtgttacaggcggggtttttcttgttgacaagaatcctcacaataccgcagagtctagactcgtggtggacttctctcaattttctagggggatcacccgtgtgtcttggccaaaattcgcagtccccaacctccaatcactcaccaacctcccgtcctccaatttgtcctggttatcgcgggatgtgtctgcggcgttttatcatattcctcttcatcctgctgctatgcctcatcttcttattggttcttctggattaccaaggtatgttgcccgtttgtcctctaattccaggatcaacaacaaccagtacgggaccctgcaaaacctgcacgactcctgctcaaggcaactctatgtttccctcatgttgctgtacaaaacctacggatggaaattgcacctgtattcccatcccatcatcttgggctttcgcaaaatacctatgggagtgggtctcagtccgtttctcttggctcagtttactagtgccatttgttcagtggttcgtagggctttcccccactgcttggctttcagctatatggatgatgtggtattgggggccaagtctgtacaacatcttgagtccctttataccgctgttaccaattttcttttgtctttgggtatacatttaaaccctaacaaaacaaagagatggggttattccctaaacttcatgggatatgtaattggaagttggggtacgttgccacaggatcatattgtacaaaaaatcaaatgctgttttagaaaacttcctgtcaatcgacctattgattggaaagtatgtcaaagaattgtgggtcttttgggctttgccgctccatttacacaatgtggttaccctgccttaatgcctttgtatgcatgtatacaagcaaaacaggcttttactttctcgccaacttacaaggcctttctaagtcaacagtatatgaacctttaccccgttgcccggcaacggccttggctatcggccatcagcgcatgagtggaacctttgtggctcctctgccgatccatactgcggaactcctagctgcttgttttgctcgcagcaggtctggagcaaaactcatcgggactgataattctgtcgtcctttctcggaaatatacatcatttccatggctgctaggttgtactgccaactggattcttcgcgggacgtcctttgtttacgtcacccctcgcggggccgcttgggactctatcgtccccttctccgtctgccgtaccgtcccggttgcatggagaccaccgtgaacgcccatcagatcctcccagctacttcaaagactgtgttaggttaaaggtctttgtattcatcatgcaactttttcacctctgcctaatcatcacttgtacatgtcccactcatggctttgggacatggacattgacccttataaagaatttggagctactgtggagttactctcatttttgccttctgacttctttccttcagtccgggatctacttgatacagcttcagctctgtatcgggaagccttagagtctccggagcattgctcccctcaccatacagcactcaggcaagccattctctgctggggggaattaatggctctagccacctgggtgggtaataatttggaagatccagcatccagggatctagtagtcaattatgttaacactaacatgggcctaaagatcagacaactattgtggtttcatatttcttgccttacttttggaagagaaactgtccttgagtatttggtctctttcggagtgtggattcgcactcctccagcctatagaccaccaaatgcccctatcctatcaacacttccggaaactactgttgttagacgacgagatcgaggcaggtcccctagaagaagaactccctcgcctcgcagacgaagatctcaatcgccgcgtcgcagaagatctcaatctcgggaatctcaatgttagtattccttggactcataaggggggaaactttactgggctttattcctctactgtccctatctttaatcctgaatggcaaactccttcttttcctaaaattcatttacatgaggacattattaataggtgtcagcaatttgtaggccctttaactgtaaatgaaaagagaagattaaaattaattatgcctgctagattttatccaaacagcaccaaatatttgcctctagacaaagggattaagccttattatcctgatcaagtagttaatcattacttccagaccagacattatttacatactctttggaaggctgggattctatataagagggaaactacacgtagcgcctcattttgcgggtcaccatattcttgggaacaagagctacatcatgggaggttggtcaacaaaacctcgcaaaggcatggggacgaatctttctgttcccaaccctctgggattctttcccgatcatcagttggaccctgcattcggagccaattcaaacaatccagattgggacttcaaccccatcaaggaccactggccacaagccaaccaggtaggagtgggagcattcgggccagggttcactcccccacacggaggtgttttggggtggagccctcaggctcagggcatattggctacagtgccaacagttcctcctcctgcctccaccaatcggcagtcaggaaggcagcctactcccatctctccacctctaagagacagtcatcctcaggccatgcagtggaaggtctgtgccaagtgtttgctgacgcaacccccactggctggggctccgtcggcgctgaatcccgcggacgcgaccacggggcgcacctctctttacgcggtctccccgtctgtgccttctcatctggcccaaggtcttacataagaggactcttggactcaatgtcaacgaccgaccttgaggctgtttaaagactgggaggagttgggggaggagataggaggctgtaggcataaattggtctgcgcaccattcaagcctccaagctgtgccttggccgtgtgcacttcgcttcacctctgcacgt

SEQ ID NO: 14 - DNA, 61 bp, Artificial
  misc_RNA
gucgugugcgcuuugcuucgccucugcuuccugucacagaggugaagcgaagugcacacgg

SEQ ID NO: 15 - DNA, 61 bp, Artificial
  misc_RNA
guacaccucgcuucaccucuacacaucuuccugucaacgugcagaggugaagcgaagugca

SEQ ID NO: 16 - DNA, 25 bp, Artificial
  Sequence encoding a target sequence of HBV genome
ccgtgtgcacttcgcttcacctctg

SEQ ID NO: 17 - DNA, 25 bp, Artificial
  Sequence encoding target sequence of HBV genome
tgcacttcgcttcacctctgcacgt

(19) United States
(12) Patent Application Publication
(10) Pub. No.: US 2008/0207539 A1
(43) Pub. Date: Aug. 28, 2008

(54) Self-Processing Rna Expression Cassette

(76) Inventors: Patrick Arbuthnot, Parktown (ZA); Marc Weinberg, Parktown (ZA); Sergio Catril Carmona, Parktown (ZA); Ely Abdullah, Parktown (ZA)

Correspondence Address: SUTHERLAND ASBILL & BRENNAN LLP, 999 PEACHTREE STREET, N.E., ATLANTA, GA, 30309, US

(21) Appl. No.: 10/568,933

(22) PCT Filed: Aug. 31, 2004
(86) PCT No.: PCT/IB04/02816

§ 371 Date: May 9, 2008

(30) Foreign Application Priority Data
Sep. 1, 2003 (ZA) 2003/6821

(51) Int. Cl. A61K 31/70; C12N 15/00; C12N 5/00; C07H 21/04

(52) U.S. Cl. 514/44; 435/320.1; 536/24.5; 435/325

(57) ABSTRACT

The invention provides a self-processing RNA expression cassette which includes at least one pair of processing units, an RNAi effecter sequence of predetermined length that regulates target gene expression which is flanked by said pair of processing units; and at least one pair of cognate ribozyme cis-cleavage target sites located 5′ and 3′ of the RNAi effecter sequence. The self-processing RNA expression cassette is able to express in vivo and in vitro and the RNAi effecter sequence includes at least one target recognition sequence derived from the Hepatitis B Virus (HBV) X gene (HBx).

THIS INVENTION relates to inhibition of viral gene expression. More specifically, this invention relates to a method of using RNA sequences to inhibit Hepatitis B Virus replication. Expression constructs containing hammerhead ribozymes and short hairpin RNAs (shRNAs) are used in the method to target specific HBV sequences.

RNA interference (RNAi) is an evolutionary conserved biological response to double-stranded RNA that has been described in plants [1], invertebrates [24] and in mammalian cells [5]. RNAi functions by directing the suppression of genes expressing homologous sequences to either endogenous or introduced double-stranded RNA (dsRNA) with no effect on genes with unrelated sequences [6, 7]. More specifically, long dsRNA is processed into shorter dsRNA (small interfering RNAs, or siRNAs) by Dicer, which is an RNase III-related nuclease [8]. siRNA fragments are typically 21-23 bp with 2 nucleotide 3′ overhangs [9] and are incorporated into a cytoplasmic RNA-induced silencing complex (RISC). RISC includes a RNA cleavage, and an RNA helicase [10] amongst other subunits [11] [12]. Using the antisense strand of siRNA as a guide sequence, RISC hybridises and cleaves target mRNA within the bound complementary region [13, 14]. Gene silencing by siRNA-mediated methylation of promoter DNA sequences has also been shown to reduce gene transcription in mammalian cells [15]. RNAi is thought to be an ancient response pathway that mediates resistance to both endogenous parasitic and exogenous pathogenic nucleic acids, and may play a role in regulating the expression of protein-coding genes [7]. Naturally occurring small RNAs function similarly to siRNAs in higher eukaryotes. These are part of a complex natural network of micro RNAs (miRNAs), which are processed by Dicer and assembled into RISC, to regulate translation of specific cellular mRNAs [16]. Processing of siRNAs by the RNAi pathway is important for the targeted degradation of ‘rogue’ viral and cellular mRNAs in mammalian cells [13, 17, 18]. The post-transcriptional silencing action of RNAi has been reported to be more efficient than either ribozyme or antisense RNA action [19].

Effecting RNAi in mammalian cells has, until recently, been a difficult undertaking. Double-stranded RNAs which are longer than 30 base-pairs trigger the non-specific interferon response pathway, which is mediated by the activation of dsRNA-dependent protein kinase (PKR) [20] and 2′,5′-oligoadenylate synthetase (2′5′OAS) [21]. This response pathway results in global repression of translation and leads ultimately to apoptosis [22]. To induce specific and significant gene silencing, intracellular delivery or production of siRNA or short hairpin RNA (shRNA) fragments of exact size is important. By introducing siRNAs as short synthetic annealed oligonucleotides (<30 bp) directly into mammalian cells, Tuschl and colleagues were successfully able to bypass the interferon pathway and effect RNAi in mammalian cell cultures [19].

Many of the studies undertaken to achieve gene silencing have used presynthesized RNAs. Typically, complementary RNA oligonucleotides are annealed in vitro to generate an exogenous source of siRNA for delivery into cells. These siRNAs may not be suitable for in vivo use. Since synthetic oligoribonucleotides are not replenished naturally within a cell, to maintain an adequate intracellular concentration for sustained activity, these molecules need to be administered regularly. Synthetic oligoribonucleotides may be chemically altered to preserve their longevity in physiological fluids. However, these modifications may have adverse toxic effects in vivo [23]. Results from a number of studies suggest that siRNAs can be expressed endogenously as independent sense and antisense RNA strands [24, 25], as shRNAs [26-30] or as derivatives of naturally-occurring miRNAs [31, 32]. Transcription of miRNA genes naturally produces pri-miRNA sequences, which are processed in the nucleus by the enzyme Drosha to form pre-miRNA. Pre-miRNA is then transported to the cytoplasm via the exportin 5 pathway, where it is processed by Dicer to form mature miRNA. Since little is known about the promoters involved in miRNA expression, most studies have used the U6 small nuclear RNA (snRNA) promoter [26] or more compact H1 promoter [7] or tRNA^{Val} promoter [33]. These promoters are recognised by RNA Polymerase III, and are capable of constitutively producing effecters of RNAi. Pol III promoters have the advantage of containing all of their control elements upstream of the transcription initiation site, and this enables the generation of expression cassettes that produce transcripts of defined length. Pol II promoters can induce tissue- or cell-type-specific RNA expression but have the disadvantage of requiring control elements downstream of the transcription initiation site. Thus in addition to potentially therapeutic RNA, additional sequences derived from regulatory elements are included in the transcript. Previous studies have shown that these additional sequences inhibit the function of siRNA molecules [34]. In fact, the silencing effect of transcribed shRNAs, or individual sense and antisense siRNA strands, is compromised by the presence of as few as 9 extra bases at the 5′ end, between the transcription start site and the 21 base pair hairpin [34]. There is at present no means of generating functional exact size shRNA or siRNA duplexes from Pol II transcripts. Chemical RNA synthesis, in vitro transcription and use of Pol III-based cassettes are currently the preferred methods of generating short RNA sequences of precise length.

Terms used herein have their art-recognised meaning unless otherwise indicated. As used herein,

Transcription

The process of producing RNA from a DNA template.

Expression Cassette

A “recombinant expression cassette” or simply “expression cassette” is a nucleic acid construct, generated recombinantly or synthetically, with nucleic acid elements which permit transcription of a particular nucleic acid in the cell. The recombinant expression cassette can be part of a plasmid, virus or nucleic acid fragment. Typically, the recombinant expression cassette includes a nucleic acid to be transcribed, and an operably linked promoter. In some embodiments, the expression cassette may also include an origin of replication and/or chromosome integration elements (e.g. a retroviral LTR).

In Silico

In silico refers to the laboratory conditions under which a reaction is carried out in a test tube (or equivalent vessel) and when no living cells are present.

In Vitro Transcription

The transcription of a DNA molecule into RNA molecules using a laboratory medium which contains an RNA polymerase and RNA precursors.

In Vivo Transcription

The transcription of a DNA molecule into RNA molecules, within a living organism.

miRNA

Micro RNAs (miRNAs) are small RNA molecules that are encoded by cellular sequences, which regulate translation of specific cellular mRNAs.

shRNA Precursor

A shRNA precursor is a hairpin RNA sequence that is processed intracellularly by Dicer to generate a shRNA molecule.

shRNA Ribozyme Pair

A shRNA ribozyme pair refers to 2 ribozymes with cis-cleavage activity at the 5′ and 3′ ends of a RNA sequence that forms a shRNA. That is, cleavage in cis by the ribozyme pair releases a RNA sequence that folds on itself to form a hairpin, which can be processed intracellularly to form a mature shRNA molecule.

Multimeric Cassette

A tandem arrangement of monomeric units.

Nucleic Acid

The term “nucleic acid” refers to deoxyribonucleotide or ribonucleotide polymer in either single- or double-stranded form, and unless otherwise limited, encompasses analogues of natural nucleotides that hybridise to nucleic acids in a manner similar to naturally occurring nucleotides. Unless otherwise indicated, a particular nucleic acid sequence includes the complementary sequence thereof.

Operably Linked

The term “operably linked” refers to a functional linkage between a nucleic acid expression control sequence (such as a promoter, enhancer or array of transcription factor binding sites) and a second nucleic acid sequence, wherein the expression control sequence directs transcription of the nucleic acid corresponding to the second sequence.

Promoter

A promoter is an array of DNA control sequences which is involved in binding of an RNA polymerase to initiate transcription of a nucleic acid. As used herein, a promoter includes necessary nucleic acid sequences near the start site of transcription, such as, in the case of a Pol II type promoter, a TATA element. A promoter also optionally includes distal enhancer or repressor elements which can be located as much as several thousand base pairs away from the start site of transcription.

Ribozyme

A molecule of ribonucleic acid (including derivatives with modified nucleotides) that has catalytic activity.

Ribozyme Cleavage in Cis

Cleavage of RNA that occurs when the catalytic and target ribozyme components are derived from a single RNA molecule (intramolecular cleavage).

Ribozyme Cleavage in Trans

Cleavage of RNA that occurs when the catalytic and target ribozyme components are derived from two RNA molecules (intermolecular cleavage.)

RNA Interference

The process by which the expression of a double stranded nucleic acid (including siRNA, shRNA) causes sequence-specific degradation of complementary RNA, sequence-specific translational suppression or transcriptional gene silencing.

RNAi-Encoding Sequence

A nucleic acid sequence which, when expressed, causes RNA interference.

shRNA

Short hairpin RNA (shRNA) is a short sequence of single stranded RNA which folds back on itself such that nucleotides from the two separate segments have base paired, and the resulting structure appears as the name describes. shRNA is a substrate for Dicer and effects RNAi (The double stranded region of the hairpin may include base mismatches i.e. non AU or GC pairs)

siRNA

Small interfering RNA (siRNA) consists of a short double-stranded RNA molecule. Typically a siRNA molecule comprises a 19 bp duplex region with 3′ overhangs of 2 nt. One strand is incorporated into a cytoplasmic RNA-induced silencing complex (RISC). This directs the sequence specific RNA cleavage that is effected by RISC. Mismatches between the siRNA guide and its target may cause translational suppression instead of RNA cleavage. siRNA may be synthetic or derived from processing of a precursor by Dicer.

Guide Sequence

A short single stranded RNA fragment derived from an RNAi effecter, for example siRNA, shRNA or shRNA that is incorporated into RISC, and which is responsible for sequence-specific degradation or translation suppression of target RNA.

RNAi Precursor

Any RNA species that is processed to form a guide sequence, which may then be incorporated into RISC and effect RNAi.

Dicer

An RNAse III enzyme, which digests double stranded RNA and is responsible for processing RNAi precursors to form siRNAs.

Processing Unit

A processing unit refers to a RNA sequence, such as a hammerhead ribozyme, which has specific endonuclease cleavage activity. Usually, a processing unit has cis cognate target sites on a transcript that also encodes an RNAi effecter sequence. Cleavage activity of the processing unit allows generation of an RNAi effecter molecule of exactly defined sequence.

RNAi Effecter

Any RNA sequence (e.g. shRNA, miRNA and siRNA) including its precursors, which can cause RNAi.

RNAi Effecter Processing Unit

RNA that includes sequences of an RNA effecter together with processing units (e.g. hammerhead ribozyme). The processing units act in cis to produce an RNAi effecter of exact sequence.

RNAi Effecter Processing Cassette

An RNAi effecter processing unit with operably linked promoter.

siRNA Ribozyme Pair

A siRNA ribozyme pair refers to 2 ribozymes with cis-cleavage activity at the 5′ and 3′ ends of a sense or antisense strand of siRNA. Cleavage in cis by the pair releases thus either the siRNA sense or antisense strand.

shRNA Ribozyme Pair

A siRNA ribozyme pair refers to 2 ribozymes with cis-cleavage activity at the 5′ and 3′ ends of a shRNA. Cleavage in cis by the pair releases shRNA.

Monomeric Unit

A nucleic acid sequence that encodes components of at least two processing units and RNAi effecter sequences. Cognate cis cleavage target sites required by the processing unit to generate shRNA or a siRNA duplex are located on the same transcript.

Subsequence

The term “subsequence” in the context of a particular nucleic acid sequence refers to a region of the nucleic acid equal to or smaller than the specified nucleic acid, or a part thereof.

Target Recognition Sequence

As used herein, the term ‘target recognition sequence’ refers to a sequence derived from a gene, in respect of which gene the invention is designed to inhibit, block or prevent gene expression, enzymatic activity or interaction with other cellular or viral factors.

This invention describes a universally applicable method, which incorporates ribozymes into expression cassettes, to allow generation of siRNA or shRNA sequences of exact size. The procedure is applicable in silico and intracellularly for the generation of RNAi effecters.

According to one aspect of the invention there is provided a self-processing or self-cleaving RNA expression cassette which includes
at least one pair of processing units,
a RNAi effecter sequence of predetermined length that regulates target gene expression which is flanked by said pair of processing units; and
at least one pair of cognate cis-cleavage target sites located at precise sites 5′ and 3′ of the RNAi effecter sequence.

The self-processing RNA expression cassette may express in vivo and/or in vitro.

In other words, broadly there is provided a self-processing or self-cleaving RNA expression cassette, which includes:

a monomeric unit selected to generate a RNAi effecter sequence, and

said expression cassette being able to express both in vivo and in vitro.

The RNAi effecter sequence may be a siRNA-encoding sequence or a shRNA-encoding sequence.

The self-processing RNA expression cassette may be a multimeric self-processing RNA expression cassette.

The RNA expression cassette may be expressed using operably linked Pol II, Pol III or bacteriophage promoters.

The monomeric unit may include hammerhead ribozymes.

The self-processing RNA expression cassette may include

a first-ribozyme, or part thereof, having a first cis-cleavage specificity, the first-ribozyme or part thereof having cis-cleavage activity and including a catalytic domain and an antisense domain;

a second-ribozyme or part thereof having a second cis-cleavage specificity, the ribozyme or part thereof having cis-cleavage activity and including a catalytic domain and an antisense domain.

The first and second ribozymes may have different cis-cleavage recognition sequences including a ribozyme cleavage site which has identity or similarity to a trans-cleavage target portion of a target transcript sequence, or subsequence thereof, each different target recognition sequence being recognizable by the respective antisense domains of the first and second ribozymes, or parts thereof.

In a preferred embodiment, the RNA expression cassette may include said first and second ribozymes and at least one RNAi effecter sequence, each ribozyme having different cis-target recognition sequences and ribozyme cleavage sites.

The first and second ribozyme may also have trans-cleavage activity.

The RNA expression cassette may include any number of monomeric units.

The RNA expression cassette may include:

at least one further ribozyme pair, in addition to the first and second ribozymes; and

at least one further sequence encoding a RNAi effecter, in addition to said RNAi effecter sequence, and which differs therefrom.

The self-processing RNA expression cassette may include separate sets of sequences encoding RNAi effecter molecules that cause sequence-specific translation inhibition.

The self-processing RNA expression cassette may include separate sets of sequences encoding RNAi effecter molecules that cause sequence-specific transcriptional silencing.

The siRNA sequences or RNA precursor molecules that effect sequence-specific translation inhibition, as well as the ribozyme antisense domain trans-cleavage target recognition sequences, may include target recognition sequences derived from Hepatitis B Virus (HBV) X gene (HBx). The target recognition sequences may be derived from at least two specific sites of the HBV HBx gene.

According to another aspect of the invention there is provided an isolated nucleic acid sequence encoding the self-processing RNA expression cassette of the invention. The nucleic acid sequence may include at least one of the sequences selected from the group consisting of SEQ ID NO. 9, SEQ ID. NO. 10, SEQ ID NO. 11, SEQ ID NO. 12, SEQ ID. NO. 14 and SEQ ID NO. 15.

The nucleic acid sequence may include SEQ ID NO. 9; a nucleic acid sequence complementary to SEQ ID NO. 9; a nucleic acid sequence which hybridizes specifically to SEQ ID NO. 9; a homologous sequence of a hepadnavirus; or a nucleic acid sequence which has at least 90% sequence identity to one of said sequences.

The nucleic acid sequence may include SEQ ID NO. 10; a nucleic acid sequence complementary to SEQ ID NO. 10; a nucleic acid sequence which hybridizes specifically to SEQ ID NO. 10; a homologous sequence of a hepadnavirus; or a nucleic acid sequence which has at least 90% sequence identity to one of said sequences.

The nucleic acid sequence may include SEQ ID NO. 11; a nucleic acid sequence complementary to SEQ ID NO. 11; a nucleic acid sequence which hybridizes specifically to SEQ ID NO. 11; a homologous sequence of a hepadnavirus; or a nucleic acid sequence which has at least 90% sequence identity to one of said sequences.

The nucleic acid sequence may include SEQ ID NO. 12; a nucleic acid sequence complementary to SEQ ID NO. 12; a nucleic acid sequence which hybridizes specifically to SEQ ID NO. 12; a homologous sequence of a hepadnavirus; or a nucleic acid sequence which has at least 90% sequence identity to one of said sequences.

The nucleic acid sequence may include SEQ ID NO. 14; a nucleic acid sequence complementary to SEQ ID NO. 14; a nucleic acid sequence which hybridizes specifically to SEQ ID NO. 14; a homologous sequence of a hepadnavirus; or a nucleic acid sequence which has at least 90% sequence identity to one of said sequences.

The nucleic acid sequence may include SEQ ID NO. 15; a nucleic acid sequence complementary to SEQ ID NO. 15; a nucleic acid sequence which hybridizes specifically to SEQ ID NO. 15; a homologous sequence of a hepadnavirus; or a nucleic acid sequence which has at least 90% sequence identity to one of said sequences.

Preferably, the nucleic acid sequence may have at least 95% sequence identity to said sequence.

According to a further aspect of the invention there is provided a nucleic acid sequence encoding a target sequence, wherein the nucleic acid sequence is CCGTGTGCACTTCGCTTCACCTCTG; a complementary nucleic acid sequence; a nucleic acid sequence which hybridizes specifically to said sequence; or a nucleic acid sequence which has at least 90% sequence identity to one of said sequences.

The nucleic acid sequence may have at least 95% sequence identity to said sequence.

According to a further aspect of the invention there is provided a nucleic acid sequence encoding a target sequence, wherein the nucleic acid sequence is TGCACTTCGCTTCACCTCTGCACGT; a complementary nucleic acid sequence; a nucleic acid sequence which hybridizes specifically to said sequence; or a nucleic acid sequence which has at least 90% sequence identity to one of said sequences.

The nucleic acid sequence may have at least 95% sequence identity to said sequence.

According to another aspect of the invention there is provided a method of inhibiting expression of at least one target RNA transcript having at least one target recognition sequence, the method including steps of:
providing a nucleic acid sequence encoding an expression construct having a self-processing RNA expression cassette according to the invention, wherein cis-cleavage activity domains of the ribozymes recognise specific cleavage sites within the self-processing RNA expression cassette, said cis-cleavage sites being arranged within the self-processing RNA expression cassette in such a manner that the cis-cleavage activity of said ribozymes produces a RNAi effecter sequence of a pre-determined length;
   expressing the nucleic acid sequence encoding the self-processing RNA expression cassette to produce the self-processing RNA expression cassette;
   producing a RNAi effecter molecule, or precursor thereof, of pre-determined length by cis-cleavage of RNA produced from the expression cassette; and
   allowing the processed RNAi effector molecule, to contact at least one target RNA transcript, whereby the RNAi effecter molecule, directs the inhibition of expression of the target RNA transcript(s).

The step of expressing the nucleic acid sequence, the step of allowing the cleaved RNAi effecter molecule, or precursor thereof, to contact at least one target RNA transcript and the inhibition of expression of the target RNA transcript(s) may occur substantially simultaneously.

According to an embodiment of the invention there is provided a vector having incorporated therein a nucleic acid sequence encoding the self-processing RNA expression cassette of the invention.

The vector may be any suitable vector known to someone skilled in the art, e.g. a viral or non-viral vector.

According to another embodiment of the invention there is provided a composition which includes the vector of the invention and a physiologically acceptable carrier.

According to another aspect of the invention there is provided a cell which includes an RNA sequence encoding a RNAi effecter sequence or precursor according to the invention. The invention also extends to a cell including DNA encoding the RNA sequences from which, according to the invention, RNAi effecter molecules are derived.

According to a further aspect of the invention there is provided a cell which includes the vector described above.

According to another aspect of the invention there is provided a method of regulating the expression of DNA, the method including the steps of:

introducing into a cell a vector having incorporated therein a nucleic acid sequence encoding a self-processing RNA expression cassette of the invention, wherein a RNAi effecter sequence, or sub-sequence thereof, recognises at least one target RNA transcript containing at least one target recognition sequence or subsequence thereof; and

causing the vector to express the nucleic acid sequence encoding the self-processing RNA expression cassette, whereby, upon expression, the RNA cassette or subsequence thereof is cleaved into its RNAi effecter, and whereby the processed RNAi effecter recognises the target RNA transcript, thereby inhibiting the expression of the target sequence or subsequence thereof.

According to another aspect of the invention there is provided a method of inhibiting the in vivo expression of DNA, the method including the steps of:

introducing a vector within an organism, wherein the vector has incorporated therein a nucleic acid sequence encoding a self-processing RNA expression cassette in accordance the invention, wherein a RNAi effecter, or subsequence thereof, recognises at (east one target RNA transcript containing at least one target recognition sequence or subsequence thereof comprising an RNA interference recognition site; and

causing the vector to express the nucleic acid sequence encoding the self-processing RNA expression cassette or subsequence thereof, whereby, upon expression, the RNA cassette or subsequence thereof is cleaved into its RNAi effecter precursor sequence, and whereby the RNAi effecter recognises the target RNA transcript, thereby inhibiting expression of the target sequence.

According to another aspect of the invention there is provided a method of inhibiting the in vivo expression of DNA, the method including the steps of:

introducing a vector within an organism, wherein the vector has incorporated therein a nucleic acid sequence encoding a self-processing RNA expression cassette in accordance the invention, wherein a RNAi effecter, or subsequence thereof, recognises at least one target DNA sequence containing at least one target recognition sequence or subsequence thereof comprising an inhibition recognition site; and

causing the vector to express the nucleic acid sequence encoding the self-processing RNA expression cassette or subsequence thereof, whereby, upon expression, the RNA cassette or subsequence thereof is cleaved into its RNAi effecter precursor sequence, and whereby the RNAi effecter inhibits transcription from the target sequence.

According to another aspect of the invention there is provided a method of inhibiting the in vitro expression of DNA, the method including the steps of:

introducing a vector within a cell, wherein the vector has incorporated therein a nucleic acid sequence encoding a self-processing RNA expression cassette or subsequence thereof according to the invention, wherein a RNAi effecter sequence, or subsequence thereof, recognises at least one target RNA transcript containing at least one target recognition sequence or subsequence thereof comprising an RNA interference recognition site; and

causing the vector to express the nucleic acid sequence encoding the self-processing RNA expression cassette or subsequence thereof, whereby, upon expression, the RNA cassette or subsequence thereof is cleaved into a RNAi effecter precursor sequence, and whereby the RNAi effecter, recognises the target RNA transcript, thereby inhibiting expression of the target sequence.

The multimeric self-processing RNA expression cassette may include any number of monomeric units.

The target recognition sequence may be derived from the HBx open reading frame of Hepatitis B Virus (HBV). More specifically, the target recognition sequence of the RNAi effecter sequence may be derived from at least two regions located within the HBx open reading frame of HBV.

According to a further aspect of the invention, there in provided the use of a self-processing RNA expression cassette as described herein in the manufacture of a preparation for treating Hepatitis B Virus (HBV) infection, or diseases caused thereby.

According to another aspect of the invention, there is provided a substance or composition for use in a method of treating Hepatitis B virus (HBV) infection, or diseases caused thereby, said substance or composition including a self-processing RNA expression cassette as described herein, and said method including administering a therapeutically effective amount of said substance or composition.

According to a further aspect of the invention there is provided a method of treating Hepatitis B Virus (HBV) infection, or diseases caused thereby, said method including administering to a subject a therapeutically effective amount of a self-processing RNA expression cassette in accordance with the invention.

According to another aspect of the invention there is a method of regulating the expression of DNA, the method including the steps of:
generating in silico a self-processing RNA expression cassette sequence according to the invention to produce a RNAi effecter by;
allowing the processing units to recognise in silico at least one target RNA transcript having at least one target recognition sequence;
self-processing of the RNA expression cassette into its individual processing units and RNAi effecter molecules;
separating or at least partially purifying the RNAi effecter; and
introducing the separated RNAi effecter into a cell whereupon it is processed intracellularly to form a mature guide sequence which then acts on the target RNA transcript, thereby inhibiting the expression of the target sequence or subsequence thereof.

The invention will now be described, by way of non-limiting example, with reference to the accompanying drawings, sequence listings and examples. In the drawings,

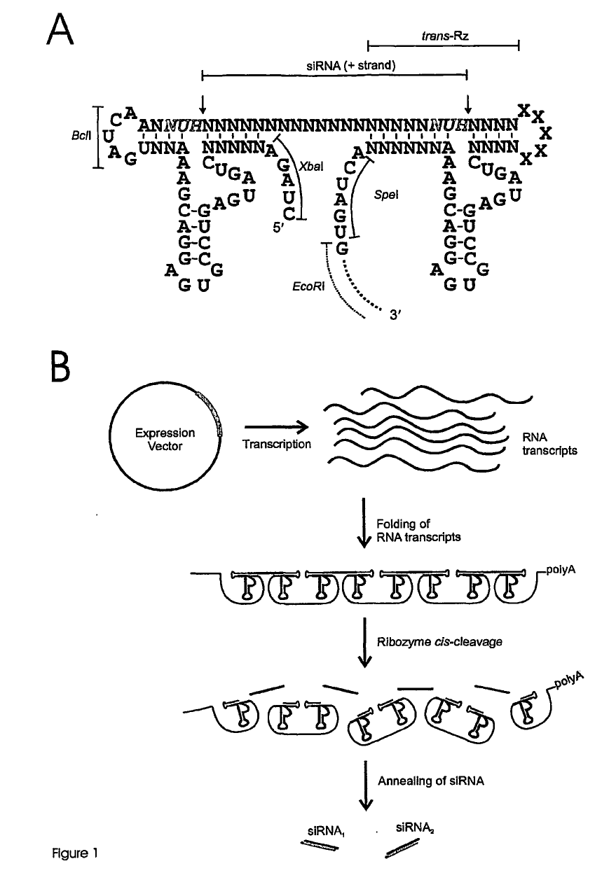

FIG. 1 shows a diagrammatic illustration of the Ribozyme-siRNA (Rz-siRNA) expression system used to generate short complementary RNA fragments that constitute the siRNA duplex. Ribozymes cleave 3′ of 5′ NUH 3′ triplets to generate the sense or antisense strands of a siRNA duplex (FIG. 1A). In this example, unique XbaI, SpeI and EcoRI restriction sites which facilitate restriction digestion and ligation of DNA fragments were included in the oligodeoxynucleotides used during the cloning procedure to generate the ribozyme/siRNA encoding sequences. A BclI site is included to aid in the screening of insert-bearing clones. Nucleotides shaded in green can optionally serve as helix I and III arms for a hammerhead ribozyme that is capable of cleaving target RNA independently in trans. In silico or intracellular transcription of cassettes yields RNA molecules that fold into ribozymes and cleave in cis to yield complementary 21 nt strands that associate to form siRNA duplexes (FIG. 1B).

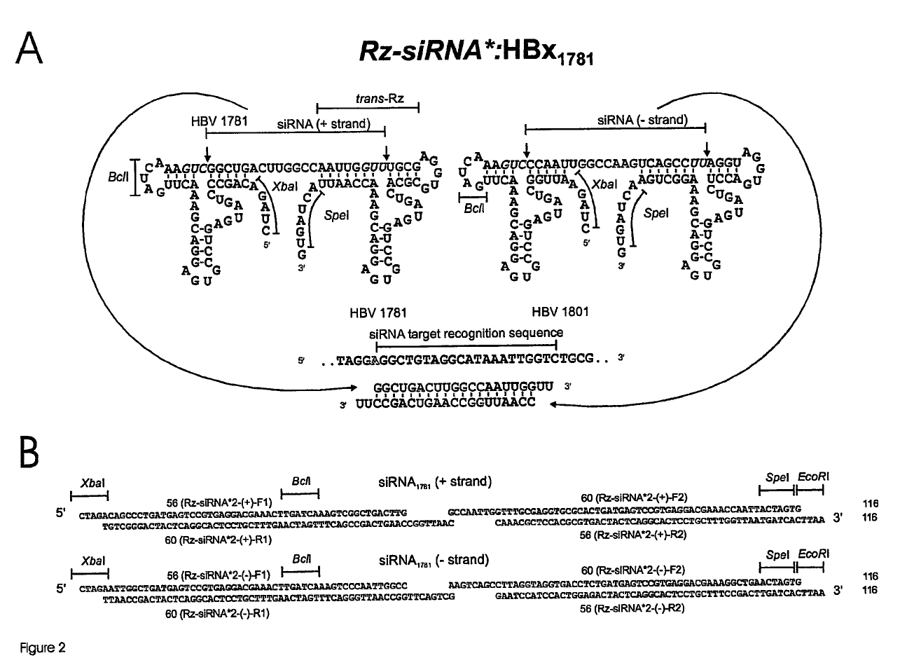

FIG. 2 shows the complete Rz-siRNA system. In total, four ribozymes cleave in cis to release + and − strands of the siRNA duplex (FIG. 2A). Sequences encoding the Rz-siRNA system that targets the HBV sequences from 1781 to 1801 are depicted. Oligonucleotides that encode ribozymes, + and − strands, together with cohesive overlaps, used to generate the complete Rz-siRNA system are depicted in FIG. 2B.

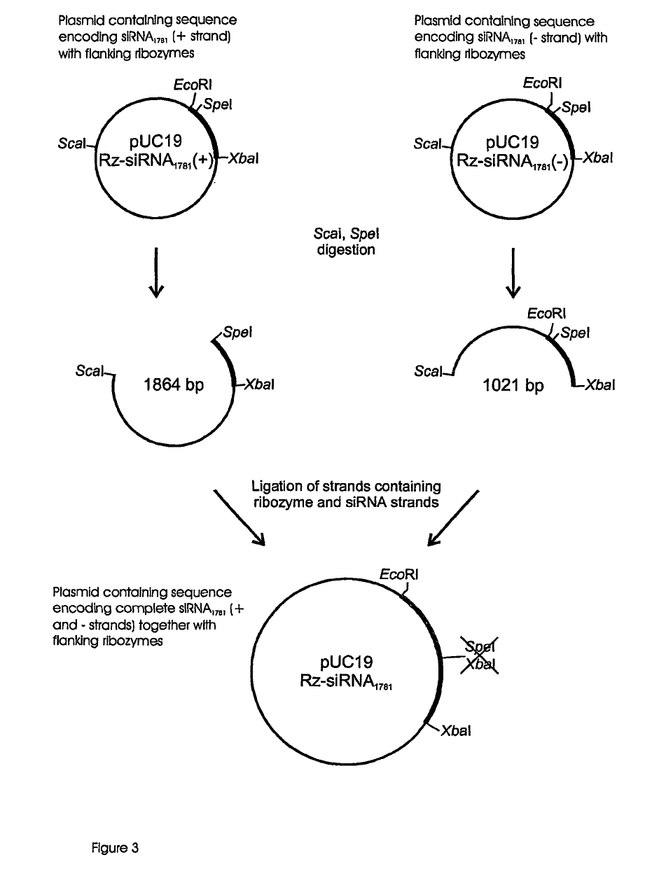

FIG. 3 shows the cloning strategy for the insertion of sequences encoding Rz-siRNA₁₇₈₁(+) or Rz-siRNA₁₇₈₁(−) into the bacterial plasmid vector pUC19 to generate pUC19 Rz-siRNA₁₇₈₁. The sets of oligonucleotides code for either the sense or antisense components of the Rz-siRNA system. Complementary single stranded overhangs of primer pairs (F1/R1 and F2/R2) enable their ligation before insertion into XbaI and EcoRI sites of pUC19 (FIG. 3). To combine sense and antisense expression systems, the sense carrying vector was digested with the ScaI and SpeI restriction enzymes whereas the antisense carrying vectors were digested with ScaI and XbaI. The 1864 bp fragment from pUCRz-siRNA₁₇₈₁(+) and the 1021 bp fragment from pUCRz-siRNA₁₇₈₁(−) were ligated together to form pUC-Rz-siRNA₁₇₈₁, which encodes both sense and antisense strands of the siRNA duplex together with ribozymes that flank each of these strands.

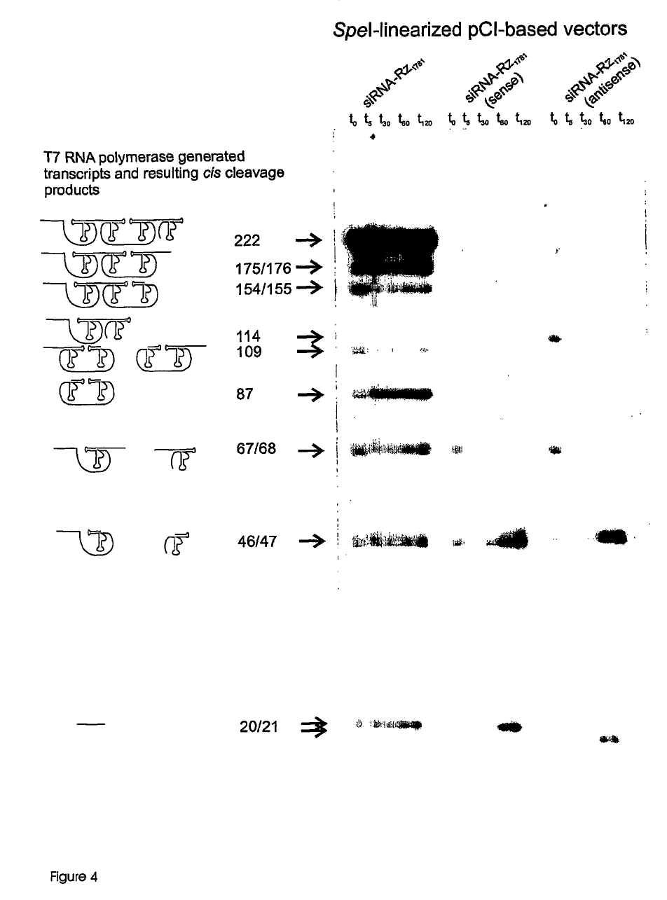

FIG. 4 shows an autoradiograph of a denaturing polyacrylamide gel in which ribozyme and siRNA fragments and fragment intermediates of radiolabelled self-processing multiribozyme transcript RNA were resolved. Products of cleavage in cis of ribozyme and siRNA molecules after transcription in vitro is depicted. pCIneo-Rz-siRNA₁₇₈₁(+), pCIneo-Rz-siRNA₁₇₈₁(−) and pCIneo-Rz-siRNA₁₇₈₁ plasmids were linearised then used to generate RNA with T7 RNA Polymerase. To the left of the autoradiograph are illustrations of the fragment intermediates predicted for each corresponding band. RNA molecules include ribozymes with precise cis-cleavage activity. Complete cleavage by 5′ and 3′ cis-ribozymes yields 20/21 nt strands that associate to form siRNA duplexes capable of effecting RNAi.

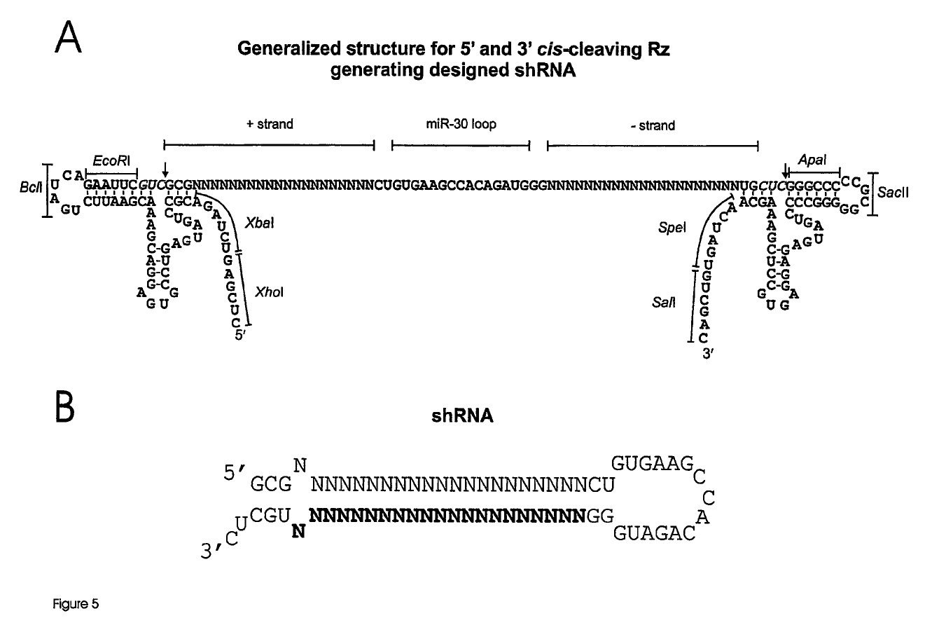

FIG. 5 shows a generic template for the production of shRNA sequences. Oligodeoxynucleotides were designed which include 5′- and 3′-flanking hammerhead ribozymes that were designed to cleave 3′ of 5′ NUH 3′ triplets (red) (FIG. 5A) to generate a shRNA (FIG. 5B). In this example the shRNA contains signature miR-30 loop and stem base regions. Mismatches may be incorporated into the stem region of the hairpin to facilitate incorporation of the appropriate single stranded RNA guide sequences (antisense) into RISC.

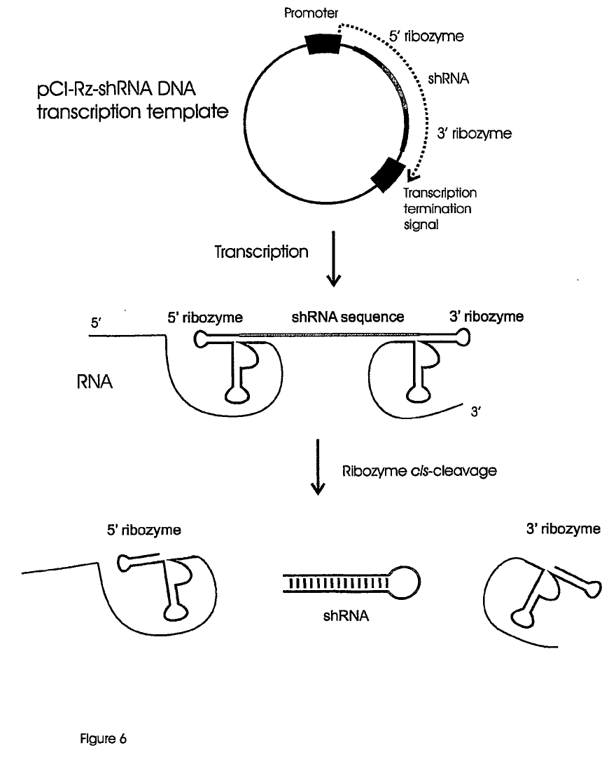

FIG. 6 shows a schematic representation of the ribozyme and shRNA expression cassettes within the eukaryotic expression plasmid vector pCIneo.

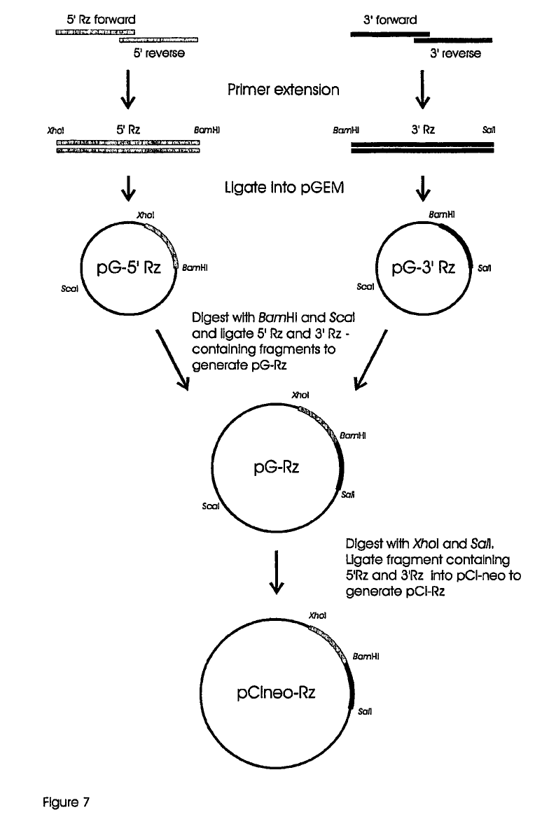

FIG. 7 depicts the strategy that was used to generate plasmid vectors that encode 5′ and 3′ ribozymes (pG-Rz and pCIneo-Rz) and which allows for subsequent insertion of an intervening shRNA-encoding sequence.

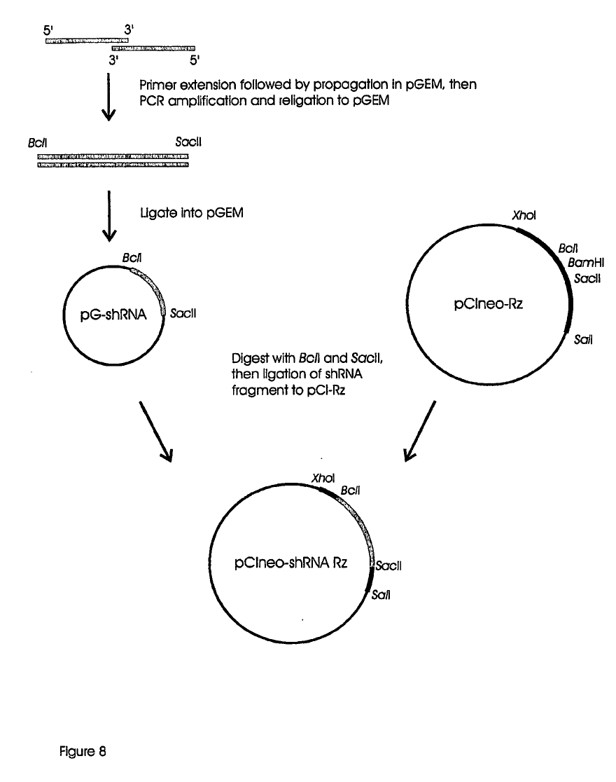

FIG. 8 illustrates the strategy that was used to insert hairpin-encoding sequences between 5′ and 3′ hammerhead ribozyme-encoding sequences to generate plasmid vectors that encode 5′ and 3′ ribozymes with an intervening shRNA-encoding sequence (pCIneo-shRNA Rz).

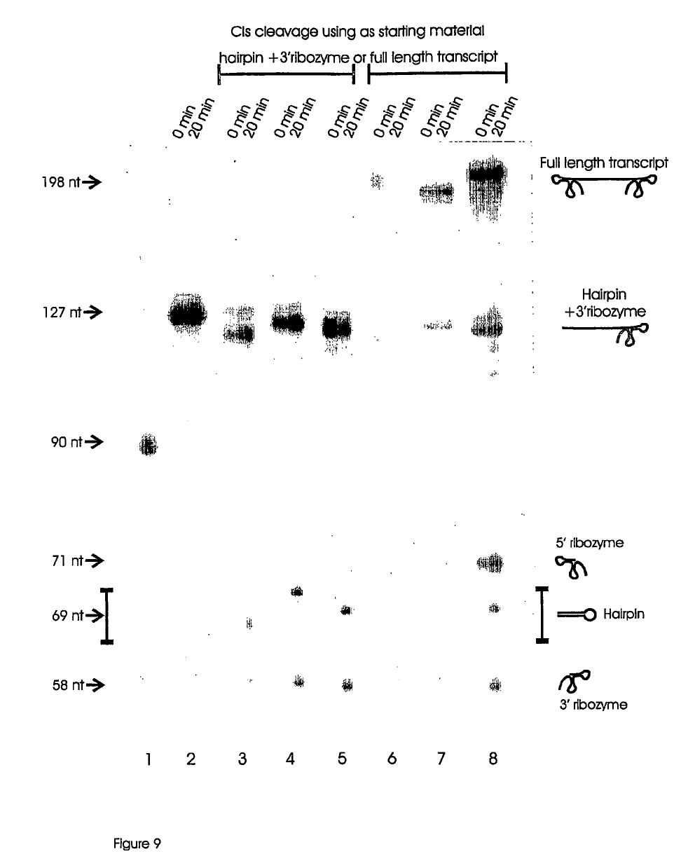

FIG. 9 shows an autoradiograph of a denaturing polyacrylamide gel on which ribozyme and shRNA fragments and fragment intermediates of radiolabelled self-cleaving multiribozyme transcript RNA were resolved. Products of cleavage in cis of ribozyme and siRNA molecules after transcription in vitro is depicted. pCIneo-Rz, pCIneo-Rz-shRNA1, pCIneo-Rz-shRNA2 and pCIneo-Rz-shRNA3 plasmids were linearised then used to generate RNA with the Riboprobe® Combination System—T3/T7 RNA Polymerase (Promega, WI, USA). To the right of the autoradiograph are illustrations of the fragment intermediates predicted for each corresponding band. RNA molecules include ribozymes with precise cis-cleavage activity. A transcript of known length (90 nt) was used as marker (lane 1). Products of the cis-cleavage reactions for pCIneo-Rz (lane 2), pCIneo-Rz-shRNA1 (lanes 3 and 6), pCIneo-Rz-shRNA2 (lanes 4 and 7) and pCIneo-Rz-shRNA3 (lanes 5 and 8) are shown. Lanes 3, 4 and 5 demonstrate cis-cleavage using an intermediate (hairpin plus 3′ ribozymes) whereas lanes 2, 6, 7, and 8 demonstrate cis-cleavage using the full length transcript. In lane 2, the single dominant band represents the transcript that includes the 5′ ribozyme and the 3′ ribozyme of pCIneo-Rz, without an intervening hairpin encoding sequence, which does not undergo cis cleavage.

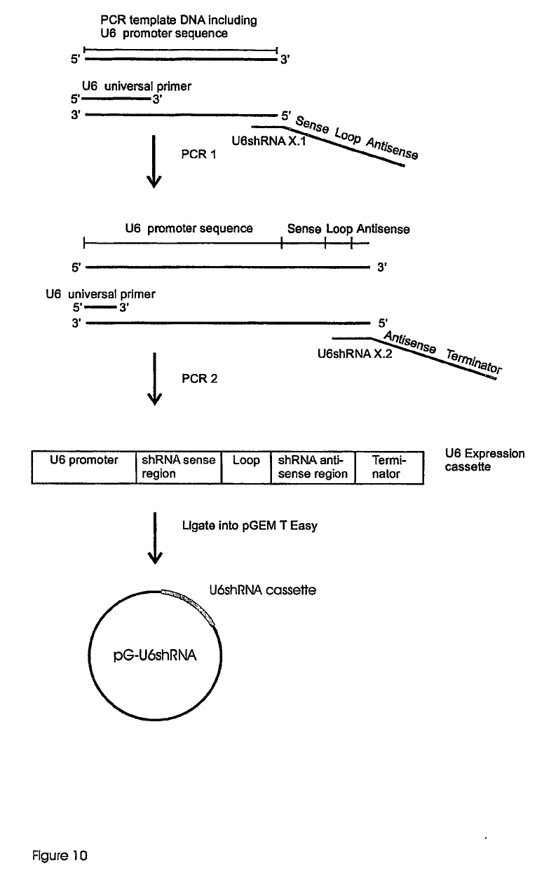

FIG. 10 shows the schematic outline of the procedure involving 2 PCR steps, which was used to generate the cassettes comprising the U6 promoter together with shRNA hairpin-encoding sequence.

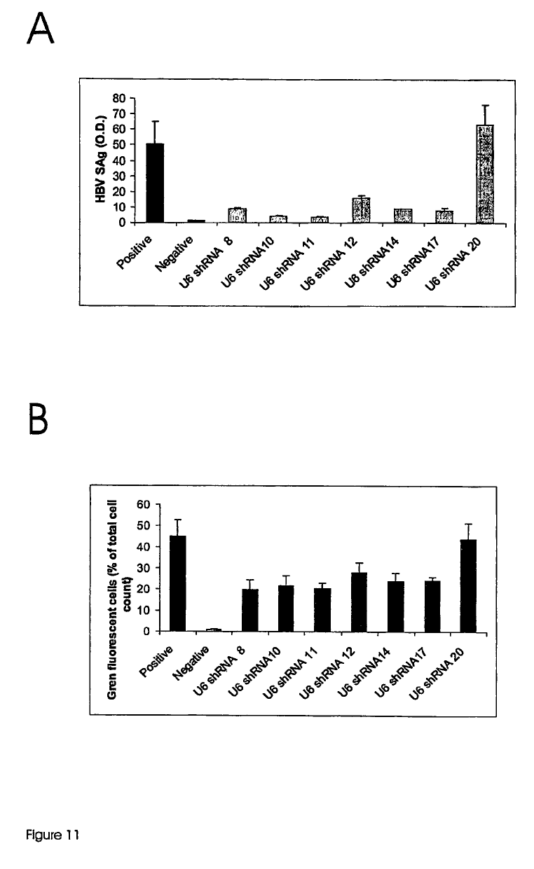

FIG. 11A shows a representative sample of the effects of pG-U6shRNA plasmids on HBsAg secretion from Huh7 cells when cotransfected with pCH-9/3091 HBV target vector [37]. In a similar series of investigations, the pCH EGFP reporter plasmid was used as target [38]. The efficacy of shRNA-encoding plasmids was assessed using flow cytometry to detect EGFP expression. The results from these investigations were similar to those obtained when using the intact HBV target plasmid (pCH-9/3091) and are depicted in FIG. 11B.

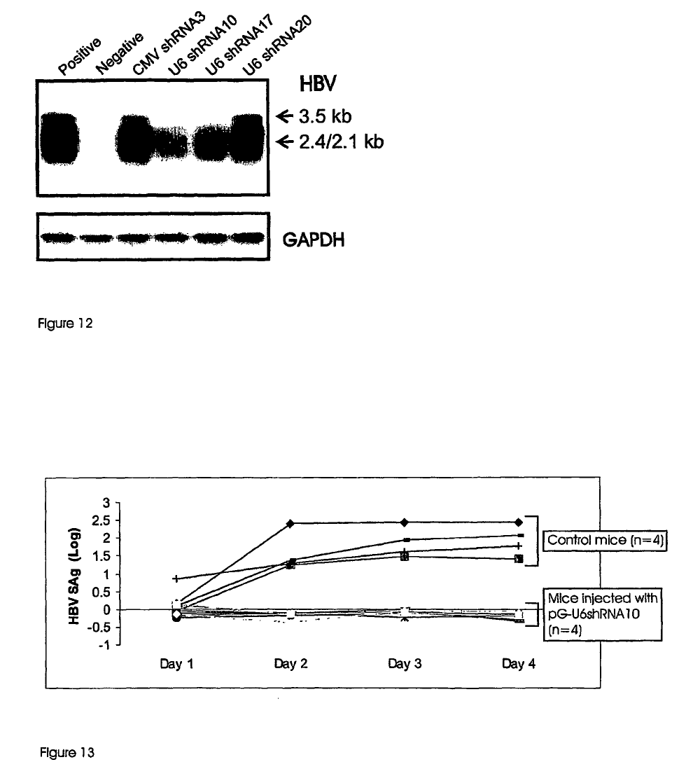

FIG. 12 shows the effects of pG-U6shRNA plasmids on HBV RNA levels in Huh7 cells when cotransfected with pCH-9/3091 HBV target construct, as measured using northern blot hybridisation of RNA extracted from transfected Huh7 cells. The blot was probed with a radiolabelled HBV sequence probe from the surface region (top panel). Cotransfected shRNA-encoding plasmids are indicated above each lane. The 3.5 kb and 2.4/2.1 kb HBV RNA transcripts that were detected from the transfected cells are indicated. As a control for the blotting efficiency and equal loading of RNA samples, the same blot was stripped and rehybridized to a GAPDH probe (FIG. 12, lower panel).

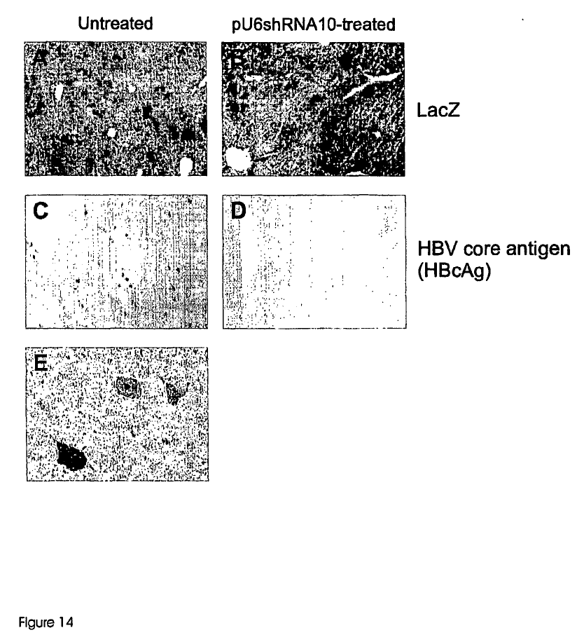

FIG. 13 shows the concentration of HBsAg over a period of 4 days in the serum of mice injected with pG-U6shRNA10, pCH3091 and LTR LacZ-encoding plasmid. Each curve represents the analysis from a single animal. Results from investigation of 4 control animals, which were injected with plasmid lacking the U6shRNA10 cassette are indicated. Four mice, which received pG-U6shRNA10 via tail vein injection, are also shown.

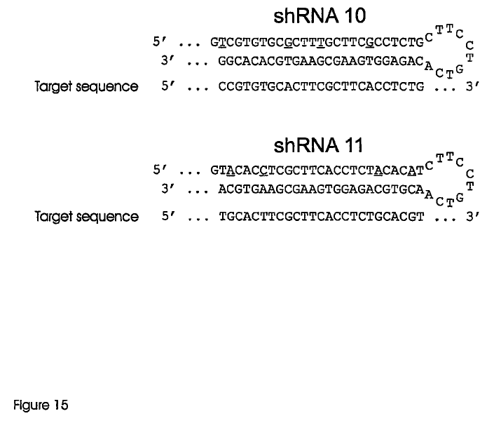

FIG. 14 shows micrographs of liver sections from the mice of FIG. 13. The liver sections were stained for β-galactosidase activity to control for the efficiency of DNA delivery. Counts of β-galactosidase-positive cells in control and pG-U6shRNA10 treated animals revealed that similar numbers of cells were transfected. Thus, equivalent and efficient expression of plasmids was achieved via the tail veins (FIGS. 14 A&B). Detection of hepatic HBV core antigen (HBcAg) using standard immunohistochemical procedures confirmed inhibition by pG-U6shRNA10 of the HBV antigen production in these liver sections (FIGS. 14 C&D). HBcAg was not detectable after co-injection with pG-U6shRNA10 (FIG. 14 D). FIG. 14 E is a representative high power field of a similar sample to that shown in FIG. 14C, which shows specific immunohistochemical staining for HBcAg. Taken together, these data demonstrate that pG-U6shRNA10 effects substantial inhibition of HBV gene expression.

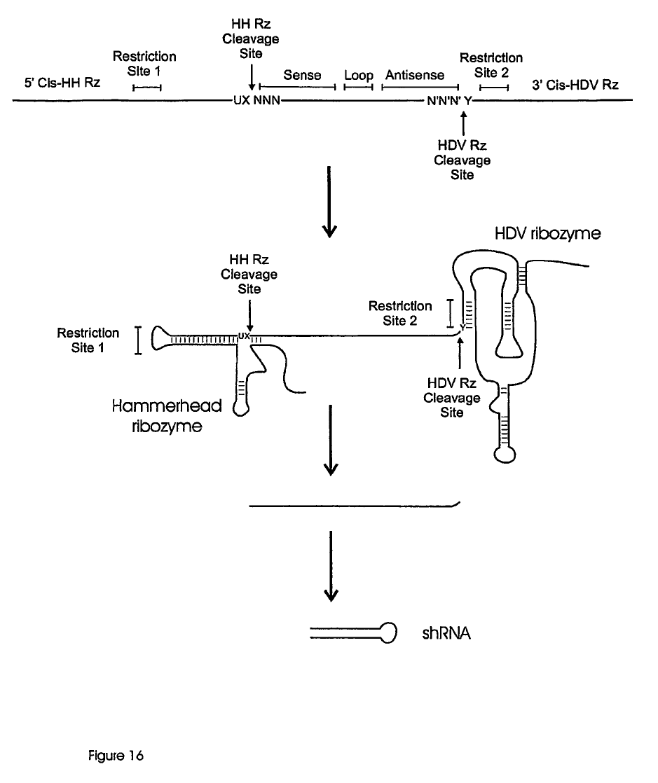

FIG. 15 shows the sequences of the effective anti-HBV shRNAs (shRNA 10 and shRNA 11), together with their HBV targets.

FIG. 16 shows a schematic illustration of the use of a combination of hammerhead and hepatitis delta virus (HDV) ribozymes to generate a shRNA sequence. Both ribozymes cleave in cis. Unlike with the combination of two hammerhead ribozymes, inclusion of a HDV ribozyme at the 3′ end allows its use as a universal cis cleaving ribozyme that could be used for generating any shRNA. Sequence requirements for HDV to be active are 3′ to the site of cleavage, and would be independent of the sequences incorporated into the hairpin.

SEQ. ID. NO. 1: Oligonucleotide sequence of the F1+ primer encoding the 5′ cis-cleaving ribozyme and part of the sense strand of the siRNA duplex for Rz-siRNA₁₇₈₁(+) that targets the HBV coordinates 1781-1801. F1+ is complementary to R1+ and includes a single stranded 5′ overhang to create a XbaI sticky end for cloning into the pUC 19 plasmid.

SEQ. ID. NO. 2: Oligonucleotide sequence of the R1+ primer that has a complementary sequence to F1+ and includes a 5′ overhang to enable ligation to the F2+/R2+ oligonucleotide duplex.

SEQ. ID. NO. 3: Oligonucleotide sequence of the F2+ primer encoding part of the sense strand of the siRNA duplex for Rz-siRNA₁₇₈₁(+) that targets the HBV coordinates 1781-1801 as well as 3′ ribozyme. F2+ is complementary to R2+ and includes a single stranded 5′ overhang to enable ligation to the F1+/R1+ oligonucleotide duplex.

SEQ. ID. NO. 4: Oligonucleotide sequence of the R2+ primer encoding a complementary sequence to F2+. A 5′ overhang generates an EcoRI sticky end for insertion into pUC19.

SEQ. ID. NO. 5: Oligonucleotide sequence of the F1− primer encoding the 5′ cis-cleaving ribozyme and part of the antisense strand of the siRNA duplex for Rz-siRNA₁₇₈₁(−) that targets the HBV coordinates 1781-1801. F1− is complementary to R1− and includes a single stranded 5′ overhang to create a XbaI sticky end for cloning into the pUC 19 plasmid.

SEQ. ID. NO. 6: Oligonucleotide sequence of the R1− primer that has a complementary sequence to F1− and includes a 5′ overhang to enable ligation to the F2−/R2− oligonucleotide duplex.

SEQ. ID. NO. 7: Oligonucleotide sequence of the F2− primer encoding part of the antisense strand of the siRNA duplex for Rz-siRNA₁₇₈₁(−) that targets the HBV coordinates 1781-1801 as well as 3′ ribozyme. A 5′ overhang is also included to enable ligation of F2−/R2− to the F1−/R1− oligonucleotide duplex.

SEQ. ID. NO. 8: Oligonucleotide sequence of the R2− primer encoding a complementary sequence to F2−. A 5′ overhang generates an EcoRI sticky end.

SEQ. ID. NO. 9: Sequence of the self-processing RNA expression cassette encoding ribozyme-shRNA 1 targeting HBV coordinates 1514-1538.

SEQ. ID. NO. 10: Sequence of the self-processing RNA expression cassette encoding ribozyme-shRNA 2 targeting HBV coordinates 1575-1599.

SEQ. ID. NO. 11: Sequence of the self-processing RNA expression cassette encoding ribozyme-shRNA 3 targeting HBV coordinates 1863-1887.

SEQ. ID. NO. 12: Sequence of the self-processing RNA expression cassette encoding siRNA-Rz1: 1781.

SEQ. ID. NO. 13: Sequence of the HBV genome AY233287A. For shRNA 10, the target sequence is depicted in bold red font, and the target of shRNA11 is in italicised red font. The overlapping regions of the two targets are bold and in italics. Sequences shown in bold black font are those targeted by other shRNAs of the panel, which were found to be less effective inhibitors of markers of HBV gene expression than shRNA 10 and shRNA 11.

SEQ. ID. NO. 14: Sequence of shRNA 10 that targets the HBV genome AY233287A.

SEQ. ID. NO. 15: Sequence of shRNA 11 that targets the HBV genome AY233287A.

Double headed arrows indicate the sites of cis cleavage by ribozymes that are positioned at the 5′ and 3′ ends of the shRNA hairpin (shRNA ribozyme pair).

Colour key for SEQ. ID. NOS. 9-12:

| | |
|---|---|
| BLACK- | STUFFER SEQUENCES |
| BLUE- | 5′ CIS CLEAVING RIBOZYME |
| GREEN- | shRNA SENSE STRAND |
| RED- | miR-30 LOOP |
| PURPLE- | shRNA ANTISENSE STRAND |
| TURQUOISE- | 3′ CIS CLEAVING RIBOZYME |

The invention described herein relates broadly to a nucleotide sequence for an expression cassette construct that encodes several units of hammerhead ribozymes and siRNA or shRNA strands. Each unit comprises two hammerhead sequences and their downstream cognate cis cleavage targets. A shRNA sequence or a siRNA sense or antisense strand encoding sequence is situated between the ribozymes. The precise cis-cleavage activity of the ribozymes allows cis-cleavage of the transcribed RNA into individual ribozyme and shRNA or siRNA strand components, the resultant shRNA or siRNA strands being of predetermined or required functional length.

Furthermore, this invention relates to a nucleic acid transfer-based approach to the inhibition of gene expression, more specifically to inhibit HBV replication. The invention also relates to a DNA sequence that encodes individual siRNA duplexes or shRNA that target specific sites on the HBx open reading frame (ORF) of HBV. The DNA sequence is designed to be included in a eukaryotic expression cassette for the expression of a multi-ribozyme-siRNA precursor RNA or multi-ribozyme-shRNA precursor RNA transcript from an operably linked Pol I, II or III promoter.

A further use of this invention is the expression of the said construct in a prokaryotic system or in silico. Therefore, the DNA sequence is also designed to be included in a prokaryotic expression cassette for the expression of a multi-ribozyme-siRNA precursor RNA or multi-ribozyme-shRNA precursor RNA transcript from operably linked bacteriophage promoters such as SP6, T3 or T7, to enable the generation and purification of siRNA or shRNA precursors in silico.

In a separate embodiment of this invention, a template expression cassette includes unique restriction cleavage sites for the incorporation of sequences that encode a shRNA precursor. A description of this ribozyme template is illustrated in FIG. 5. A further use of this embodiment is transcription from operably linked bacteriophage promoters such as SP6, T3 and T7, to enable the generation of siRNA or shRNA precursors in vitro.

### EXAMPLE 1

### Design and Propagation of Combination siRNA and Ribozyme-Expressing Constructs

1. Generation of Cassettes Encoding Ribozyme and siRNA Sequences that Target HBV

These methods describe the preparation of constructs encoding 5′ and 3′ cis-acting hammerhead ribozymes that flank sense and antisense sequences of siRNA targeted to the HBx open reading frame of the Hepatitis B Virus (HBV coordinates 1781 to 1801) (FIG. 2).

Expression cassettes that produce both strands of anti HBV siRNA were designed with a hammerhead ribozyme present on each of the 5′ and 3′ ends of both sense and antisense components of the duplex (FIG. 2). The sequence directed to HBV coordinates 1781-1801 were chosen according to guidelines developed by Elbashir and colleagues [13]. Oligodeoxynucleotides encoding the sense and antisense siRNA sequences were synthesised using phosphoramadite-chemistry (Roche Biotechnologies, Germany). Sets of four oligonucleotides encoded the sense or antisense strands with cis-cleaving ribozymes (FIG. 2B). A siRNA expression cassette was therefore derived from eight oligonucleotides that encoded four cis ribozymes and the sense and antisense strands of the siRNA duplex. The sequences of the oligonucleotides encoding the siRNA cassettes that target the HBV coordinates 1781-1801 were:

| | |
|---|---|
| F1+ | 5′-CTAGACAGCCCTGATGAGTCCGTGAGGACGAAACTTGATCAAAG |
| | TCGGCTGTAGGC-3′, |
| R1+ | 5′-CAATTTATGCCTACAGCCGACTTTGATCAAGTTTCGTCCTCACG |
| | GACTCATCAGGGCTGT-3′, |
| F2+ | 5′-ATAAATTGGTTTGCGAGGTGCGCACTGATGAGTCCGTGAGGACG |
| | AAACCAATTACTAGTG-3′, |
| R2+ | 5′-AATTCACTAGTAATTGGTTTCGTCCTCACGGACTCATCAGTGCG |
| | CACCTCGCAAAC-3′ |

for Rz-siRNA₁₇₈₁(+)
and

| | |
|---|---|
| F1− | 5′-CTAGAATTGGCTGATG AGTCCGTGAGGACGAAACTTGATCAAA |
| | GTCCCAATTTATG-3′, |
| R1− | 5′-GCTGTAGGCATAAATTGGGACTTTGATCAAGTTTCGTCCTCACG |
| | GACTCATCAGCCAATT-3′, |
| F2− | 5′-CCTACAGCCTTAGTAGGTGACCTCTGATGAGTCCGTGAGGACGA |
| | AAGGCTGTACTAGTG-3′, |
| R2− | 5′-AATTCAC TAGTACAGCCTTTCGTCCTCACGGACTCATCAGAGG |
| | TCACCTACCTAAG-3′ |

for Rz-siRNA₁₇₈₁(−).

1.1 Generation of Ribozyme and siRNA-Encoding Expression Cassettes and Cloning into pUC19 and pCI-neo Vectors

Sequences encoding Rz-siRNA₁₇₈₁(+) or Rz-siRNA₁₇₈₁(−) were inserted into the bacterial cloning vector pUC19 (Promega, USA) (FIG. 3). F2+, F2−, R1+ and R1− oligonucleotides were phosphorylated with T4 polynucleotide kinase and then annealed after heating to 100° C. and cooling to room temperature. F1+ was annealed to R1+, F2+ was annealed to R2+, F1− was annealed to R1− and F2− was annealed to R2−. The double stranded oligonucleotides have complementary single stranded 5′ overhangs of 8 nucleotides that enable ligation of each of the + and − pairs. For F1+/R1+ and F2+/R2+, the single stranded overhanging nucleotides are 5′CAATTTAT3′ (from the R1+ sequence) and 5′ATAAATTG3′ (from the F2+ sequence). For the F1−/R1− and F2−/R2− pairs, the single stranded overhanging nucleotides are 5′GCTGTAGG3′ (from the R1− sequence) and 5′CCTACAGC3′ (from the F2− sequence). The approach of joining together shorter oligonucleotides was used to overcome problems that are associated with errors that arise when synthesizing long oligonucleotides (>50 mer). The resulting ligated oligonucleotide pairs have additional 5′ overhangs to allow ligation to sticky ends generated by digestion of the pUC plasmid with EcoRI (5′AATT3′ derived from R2+ and R2−) and XbaI (5′CTAG3′ derived from F1+ and F1−). The dsDNA fragments were ligated to XbaI and EcoRI sites of the pUC19 vector to generate pUCRz-siRNA₁₇₈₁(+) and pUCRz-siRNA₁₇₈₁(−) (FIG. 3). Recombinant pUC vectors containing the correct inserts were identified according to standard procedures of sequencing and restriction mapping of plasmids isolated from colonies of transformed E. coli. Sequences from pUCRz-siRNA₁₇₈₁(+) and pUCRz-siRNA₁₇₈₁(−) were combined to generate pUC-Rz-siRNA₁₇₈₁. pUCRz-siRNA₁₇₈₁(+) was digested with the restriction enzymes ScaI and SpeI to generate 1864 and 911 bp fragments, and pUCRz-siRNA₁₇₈₁(−) was digested with ScaI and XbaI to generate 1754 and 1021 bp fragments (FIG. 3). The fragments were separated on a 0.8% agarose gel and then purified. The 1864 bp fragment of pUC-Rz-siRNA₁₇₈₁(+) was ligated to the 1021 bp fragment of pUC-Rz-siRNA₁₇₈₁(−) to generate pUC-Rz-siRNA₁₇₈₁. A similar restriction and ligation strategy allows combination of DNA fragments for the generation of vectors with multiple siRNA-encoding sequences.

The ribozyme and siRNA-encoding insert from pUC-Rz-siRNA₁₇₈₁ was amplified using PCR before insertion into the mammalian expression vector pCI-neo. Primers lying 60 bp to either side of the insert was designed with the following sequences: Forward primer 5′-CGATTAAGTTGGGATACGCC-3′ and Reverse primer 5′-CACAGGAAACAGCTATGACC-3′. The insert was amplified using a standard. PCR protocol. Initial denaturation for 5 minutes at 95° C. followed by 30 cycles of heat denaturation at 95° C. for 1 minute, 30 seconds of primer annealing at 55° C. and 1 minute of primer extension at 72° C. The amplicons were digested with the restriction enzymes EcoRI and XbaI and then ligated into pCI-neo to generate pCIneo-Rz-siRNA₁₇₈₁(+), pCIneo-Rz-siRNA₁₇₈₁(−) and pCIneo-Rz-siRNA₁₇₈₁. All inserts were sequenced using standard manual and automated chain termination procedures.

1.2 Transcription of pCI Constructs Encoding Ribozyme and siRNA Sequences that Target HBV

pCIneo-Rz-siRNA₁₇₈₁(+), pCIneo-Rz-siRNA₁₇₈₁(−) and pCIneo-Rz-siRNA₁₇₈₁ plasmids were linearised with EcoRI and purified after agarose gel electrophoresis before using as a template DNA for in vitro transcription. Radiolabelled self-cleaving RNA was transcribed at 37° C. for 1 hour in a 20 μl reaction mixture containing 2 μg of template DNA, 10 mM dithiothreitol, 40 mM Tris-HCl (pH 8.0), 8 mM MgCl₂, 2 mM spermidine, 20 U RNasin (Promega, WI, USA), 8 mM ATP, 8 mM CTP, 8 mM UTP, 12.5 μM GTP (Roche, Germany) and 20 μCi of α-³²P GTP (3000 Ci/mmol; NEN du Pont, USA) and, 20 U of T7 RNA Polymerase (Promega, WI, USA). Twenty U of DNase I (Promega, WI, USA) was added to the reaction mixture for 10 min at 37° C. RNA fragments were purified using the Qiagen RNeasy (Qiagen, CA, USA) RNA purification kit according to the manufacturer's instructions. The cleavage reaction was carried out in a 40 μl reaction mixture containing radiolabelled self-cleaving multiribozyme transcript RNA. The mixture contained 20 mM MgCl₂ and 50 mM Tris-Cl (pH 8.0), and was incubated at 37° C. Aliquots (10 μl) were removed after incubation for 0 minutes, 5 minutes and 30 minutes. Samples were resolved by denaturing polyacrylamide gel electrophoresis and then subjected to autoradiography for 1 to 12 hours (FIG. 4).

### EXAMPLE 2

### Synthesis and Characterisation of Constructs Encoding shRNA and Ribozyme Sequences

2. Generation of Cassettes Encoding Ribozyme and shRNA Sequences that Target HBV

These methods describe the preparation of constructs encoding 5′ and 3′ cis-acting hammerhead ribozymes that flank a shRNA encoding sequence.

FIG. 5 shows a generic ribozyme template for the production of shRNA sequences. Oligodeoxynucleotides were designed which include 5′- and 3′-flanking hammerhead ribozymes that were designed to cleave 3′ of 5′ NUH 3′ triplets (red) (FIG. 5A) to generate shRNA (FIG. 5B), which in this example contains signature miR-30 loop and stem base regions. Mismatches may be incorporated into the stem region of the hairpin to facilitate incorporation of the appropriate single stranded guide RNA sequences (antisense) into RISC. In this example, unique XhoI and SalI restriction sites were included in the oligodeoxynucleotides to enable insertion of the oligodeoxynucleotides into Pol II or Pol III-based expression vectors. SpeI and XbaI restriction sites were included in the oligodeoxynucleotides to facilitate cloning and enable the formation of head-to-tail ribozyme and siRNA or shRNA multimers. Unique EcoRI and ApaI restriction sites were included in the oligodeoxynucleotides to allow for the insertion of specific sequences encoding siRNA or shRNA precursors. BclI and SacII sites were included in the oligodeoxynucleotides to aid in the screening of insert-bearing clones. A schematic representation of the ribozyme and shRNA expression cassettes within a eukaryotic plasmid vector (pCIneo) is depicted in FIG. 6.

2.1 Generation of Ribozyme-Encoding Constructs by Cloning into the p-GEM T Easy and pCI-Neo Vectors for Production of Ribozyme and shRNA Expression Cassettes

To generate eukaryotic ribozyme and shRNA expression cassettes that target the HBV X open reading frame (ORF), ribozyme encoding constructs, without the intervening hairpin-encoding sequences were initially constructed. Oligonucleotides were designed to encode the 5′ and 3′ ribozymes with a spacer sequence between them. Complementary oligonucleotide sequences for the 5′ ribozyme were: 5′-GATCCTCGAGTCTAGACGCCTGATGAGTCCGTGAGGACGAACGAAT-3′ (5′Rz forward) and 5′-GATCTTGGATCCTTGAATTCTGATCAGAATCGTTTCGTCCTCACGG-3′ (5′Rz reverse). Complementary oligonucleotide sequences for the 3′ ribozyme were: 5′-GATCAAGGATCCAAGGGCCCCCGCGGGGGCCCCTGATGAGAGGAGT-3′ (3′Rz forward) and 5′-GATCGTCGACACTAGTTGCTTTCGAGGCACTCCTCTCATCAGGGGC-3′ (3′Rz reverse). 5′Rz forward was annealed to 5′Rz reverse and 3′Rz forward was annealed to 3′Rz reverse. Primer extension was performed on the annealed oligonucleotides to generate a 75 nt double stranded DNA (dsDNA) encoding the 5′ ribozyme and a 74 nt dsDNA encoding the 3′ribozyme. The 75 nt dsDNA and the 74 nt dsDNA were ligated into the PCR cloning vector pGEM-T Easy (Promega, WI, USA) to generate pG-5′Rz and pG-3′Rz, respectively. pG-5′Rz and pG-3′Rz were digested with the restriction enzymes BamHI and ScaI. The fragments containing the ribozyme sequences were eluted and purified then ligated together to generate pG-Rz, which included both 5′ and 3′ ribozymes. To generate expression vectors, pG-Rz was digested with the restriction enzymes XhoI and SalI and the ribozyme dimer containing sequence was ligated to equivalent sites of the mammalian expression vector pCI-neo (Promega, WI, USA) to generate pCIneo-Rz. This cloning strategy is represented schematically in FIG. 7.

2.2 Generation of shRNA-Encoding Constructs by Cloning into the p-GEM T Easy and pCI-Neo Vectors for Production of Ribozyme and shRNA Expression Cassettes

Oligonucleotides encoding shRNAs that target specific HBV sites were designed for insertion into pCI-Rz. The sequences were:

5′GATCGAATTCGTCGCCCGCGGGGCGCACTTCTCTTCTGTGAAGCCACA
GATGG-3′ (shRNA1 forward),
5′-GATCGGGCCCGAGCAACCACGGGGCGCACCTCTCTTCCCATCTGTGG
CTTCACAG-3′ (shRNA1 reverse),
5′-GATCGAATTCGTCGCGACGTGTGTACTTCGCCCTGTGAAGCCACAGA
TGG-3′ (shRNA2 forward),
5′-GATCGGGCCCGAGCACCGTGTGCACTTCGCTTCACCCCCATCTGTGG
CTTCACAG-3′ (shRNA2 reverse),
5′-GATCGAATTCGTCGCGCTCAGGCCTCCAAGTTGTGCCCTGTGAAGCC
ACAGATGG-3′ (shRNA3 forward)
and
5′-GATCGGGCCCGAGCATTCAAGCCTCCAAGCTGTGCCCCCATCTGTGG
CTTCACAG-3′ (shRNA3 reverse).

To complete the generation of the ribozyme and shRNA cassettes, shRNA1 forward was annealed to shRNA1 reverse, shRNA2 forward to shRNA2 reverse and 5′-GATCGTCGACACTAGTTGCTTTCGAGGCACTCCTCTCATCAGGGGC-3′ (3′Rz reverse). 5′Rz forward was annealed to 5′Rz reverse and 3′Rz forward was annealed to 3′Rz reverse. Primer extension was performed on the annealed oligonucleotides to generate a 75 nt double stranded DNA (dsDNA) encoding the 5′ ribozyme and a 74 nt dsDNA encoding the 3′ribozyme. The 75 nt dsDNA and the 74 nt dsDNA were ligated into the PCR cloning vector pGEM-T Easy (Promega, WI, USA) to generate pG-5′Rz and pG-3′Rz, respectively. pG-5′Rz and pG-3′Rz were digested with the restriction enzymes BamHI and ScaI. The fragments containing the ribozyme sequences were eluted and purified then ligated together to generate pG-Rz, which included both 5′ and 3′ ribozymes. To generate expression vectors, pG-Rz was digested with the restriction enzymes XhoI and SalI and the ribozyme dimer containing sequence was ligated to equivalent sites of the mammalian expression vector pCI-neo (Promega, WI, USA) to generate pCIneo-Rz. This cloning strategy is represented schematically in FIG. 7.

2.2 Generation of shRNA-Encoding Constructs by Cloning into the p-GEM T Easy and pCI-Neo Vectors for Production of Ribozyme and shRNA Expression Cassettes

Oligonucleotides encoding shRNAs that target specific HBV sites were designed for insertion into pCI-Rz. The sequences were:

5′GATCGAATTCGTCGCCCGCGGGGCGCACTTCTCTTCTGTGAAGCCACA
GATGG-3′ (shRNA1 forward),
5′-GATCGGGCCCGAGCAACCACGGGGCGCACCTCTCTTCCCATCTGTGG
CTTCACAG-3′ (shRNA1 reverse),
5′-GATCGAATTCGTCGCGACGTGTGTACTTCGCCCTGTGAAGCCACAGA
TGG-3′ (shRNA2 forward),
5′-GATCGGGCCCGAGCACCGTGTGCACTTCGCTTCACCCCCATCTGTGG
CTTCACAG-3′ (shRNA2 reverse),
5′-GATCGAATTCGTCGCGCTCAGGCCTCCAAGTTGTGCCCTGTGAAGCC
ACAGATGG-3′ (shRNA3 forward)
and
5′-GATCGGGCCCGAGCATTCAAGCCTCCAAGCTGTGCCCCCATCTGTGG
CTTCACAG-3′ (shRNA3 reverse).

To complete the generation of the ribozyme and shRNA cassettes, shRNA1 forward was annealed to shRNA1 reverse, shRNA2 forward to shRNA2 reverse and shRNA3 forward to shRNA3 reverse. Primer extension was performed on the annealed oligonucleotides and the resulting 92 nt dsDNA fragments were ligated into the pGEM-T Easy vector to generate pG-shRNA1, pG-shRNA2 and pG-shRNA3. To add BclI and SacII restriction sites to the ends of the shRNA sequences the plasmids (pG-shRNA1, pG-shRNA2 and pG-shRNA3) were amplified using PCR with primers containing these restriction sites. The sequences of the primers were: 5′-GATCTGATCAGATCGAATTCGTCGCG-3′ for the BclI primer and 5′-GATCCCGCGGGGATCGGGCCCAGCA-3′ for the SacII primer. The resulting 112 nt amplicons were ligated into the pGEM-T Easy vector to generate pG-shRNA1*, pG-shRNA2* and pG-shRNA3*. To generate the complete ribozymes-shRNA expression system pCI-Rz, pG-shRNA1*, pG-shRNA2* and pG-shRNA3* were digested with BclI and SacII. The 102 nt fragments digested from the pG-shRNA vectors were ligated into pCI-Rz to generate pCI-Rz-shRNA1, pCI-Rz-shRNA2 and pCI-Rz-shRNA3. This cloning strategy is represented schematically in FIG. 8.

2.3 Transcription of pCI-Neo Constructs Encoding Ribozyme and shRNA Sequences that Target HBV

To assess cis cleavage of transcripts in vitro, templates were generated by linearizing plasmids pCI-Rz, pCI-Rz-shRNA1, pCI-Rz-shRNA2 and pCI-Rz-shRNA3 with restriction enzymes (SalI for T7 transcription and XhoI for T3 transcription). In vitro transcription was carried out with the Riboprobe® Combination System—T3/T7 RNA Polymerase (Promega, WI, USA) according to the manufacturer's instructions. The reactions were resolved on a 10% denaturing (8M urea) polyacrylamide gel. Full length and intermediate transcripts from in vitro transcriptions reaction were removed from the polyacrylamide gels and purified. FIG. 9 shows the results from analysis of products of cis cleavage. The reactions included MgCl₂ at a concentration of 50 mM, which is favourable for ribozyme cleavage. Two cis-cleavage reactions were undertaken in the experiments described in FIG. 9. Both the full length transcript and also an intermediate cleavage product, comprising the hairpin sequence and 3′ ribozyme, were used as the starting material for the cis cleavage reaction. Analysis of starting material (0 min.) and also products after 20 minutes of incubation was carried out. The autoradiograph in FIG. 9 shows cis-cleavage assay. A transcript of known length (90 nt) was used as marker (lane 1). Products of the cis-cleavage reactions for pCI-Rz (lane 2), pCI-Rz-shRNA1 (lanes 3 and 6), pCI-Rz-shRNA2 (lanes 4 and 7) and pCI-Rz-hRNA3 (lanes 5 and 8) are shown. Lanes 3, 4 and 5 demonstrate cis-cleavage using an intermediate (hairpin plus 3′ ribozymes) whereas lanes 2, 6, 7, and 8 demonstrate cis-cleavage using the full length transcript. In lane 2, the single dominant band represents the transcript that includes the 5′ ribozyme and the 3′ ribozyme of pCI-Rz, without an intervening hairpin encoding sequence, which does not undergo cis cleavage.

### EXAMPLE 3

### Identification of Susceptible siRNA and shRNA Targets of HBV

3.1 Generation of shRNA Expression Constructs which Include the U6 Promoter

To identify HBV sequences within the HBV X ORF that are susceptible to knockdown, a panel of 10 shRNA expression constructs under the transcriptional control of the U6 promoter (an RNA polymerase III promoter) was generated. The schematic outline of the procedure used to generate the cassettes comprising the U6 promoter together with short hairpin-encoding sequence is depicted schematically in FIG. 10. Briefly, oligonucleotides encoding the short hairpins were designed. The sequences were:

5′-TGACGTGACAGGAAGCGTTAGCAGACACTTGGCATAGGCCCGGTGTT
TCGTCCTTTCCACA-3′ (U6shRNA2.1),
5′-CCCAGATCTACGCGTAAAAAAGGTCTGTGCCAAGTGTTTGCTGACGT
GACAGGAAGCGTTA-3′ (U6shRNA2.2),
5′-GGACGTGACAGGAAGCGTTCGTGGGATTCAGCGTCGATGGCGGTGTT
TCGTCCTTTCCACA-3′ (U6shRNA6.1),
5′-CCCAGATCTACGCGTAAAAAACCGTCGGCGCTGAATCCCGCGGACGT
GACAGGAAGCGTTC-3′ (U6shRNA6.2),
5′-CTTTATGACAGGAAGCAAAGAGAGATGCGCCCCATGGCCGCGGTGTT
TCGTCCTTTCCACA-3′ (U6shRNA7.1),
5′-CCCAGATCTACGCGTAAAAAACGACCACGGGGCGCACCTCTCTTTAT
GACAGGAAGCAAAG-3′ (U6shRNA7.2),
5′-ACGCGTGACAGGAAGCGTGTGAAGAGAGGTGTGCCCTGTGCGGTGTT
TCGTCCTTTCCACA-3′ (U6shRNA8.1),
5′-CCCAGATCTACGCGTAAAAAACACGGGGCGCACCTCTCTTTACGCGT
GACAGGAAGCGTGT3′ (U6shRNA8.2),
5′-CTCGTGACAGGAAGCAGAGGCGAAGCAAAGCGCACACGACGGTGTTT
CGTCTTTCCACA-3′ (U6shRNA10.1),
5′-CCCAGATCTACGCGTAAAAAACCGTGTGCACTTCGCTTCACCTCTGT
GACAGGAAGCAGAG-3′ (U6shRNA10.2),
5′-CACGTTGACAGGAAGATGTGTAGAGGTGAAGCGAGGTGTACGGTGTT
TCGTCCTTTCCACA-3′ (U6shRNA11.1),
5′-CCCAGATCTACGCGTAAAAAATGCACTTCGCTTCACCTCTGCACGTT
GACAGGAAGATGTG-3′ (U6shRNA11.2),
5′-GGACTTGACAGGAAGAGTTCTTTTATGTAGGACTTTGGGCCGGTGTT
TCGTCCTTTCCACA-3′ (U6shRNA12.1),
5′-CCCAGATCTACGCGTAAAAAAGCCCAAGGTCTTACATAAGAGGACTT
GACAGGAAGAGTTC-3′ (U6shRNA12.2),
5′-AGGCTGACAGGAAGGCTTCAAGGTTGGTTGTTGACGTTGCGGTGTTT
CGTCCTTTCCACA-3′ (U6shRNA14.1),
5′-CCCAGATCTACGCGTAAAAAACAATGTCAACGACCGACCTTGAGGCT
GACAGGAAGGCTTC-3′ (U6shRNA14.2),
5′-TTGGTTGACAGGAAGACTAATTTGTGCCTACAGCTTCTTACGGTGTT
TCGTCCTTTCCACA-3′ (U6shRNA17.1),
5′-CCCAGATCTACGCGTAAAAAATAGGAGGCTGTAGGCATAAATTGGTT
GACAGGAAGACTAA-3′ (U6shRNA17.2),
5′-CTTGGTGACAGGAAGCCAAAGCACAACTCGGAGGCTCGAACGGTGTT
TCGTCCTTTCCACA-3′ (U6shRNA20.1)
and
5′-CCCAGATCTACGCGTAAAAAATTCAAGCCTCCAAGCTGTGCCTTGGT
GACAGGAAGCCAAA-3′ (U6shRNA20.2).

U6 shRNA X.1 primers were complementary to part of the U6 promoter and included the sense sequences of the short hairpin, together with the loop and part of the antisense sequences. U6 shRNA X.2 primers included the remainder of the short hairpin encoding cassette, which comprised part of the antisense loop and transcription termination sequence. The universal U6 primer had the sequence: 5′-CTAACTAGTGGCGCGCCAAGGTCGGGCAGGAAGAGGG-3′. The 1st step of a two-step PCR was performed with the U6shRNAX.1 serving as reverse primers and the U6 universal primer as the forward primer. A plasmid vector in which the U6 promoter had been previously inserted [36] was used as the template for amplification. The 2^{nd} step of the two-step PCR involved amplification with U6shRNA X.2 and again the U6 universal primer. Thus after completing amplification reactions using a U6 promoter template, according to the scheme outlined in FIG. 10, the entire U6 promoter and shRNA hairpin sequence was generated. The PCR products from the final amplification step were ligated into the PCR cloning vector, pGEM-T Easy to generate pG-U6shRNA plasmids (e.g. pG-U6shRNA10 and pG-U6shRNA11). The sequences of all of the fragments inserted into pGEM-T Easy were confirmed according to standard manual or automated sequencing procedures involving dideoxy chain termination reactions.

3.2 Assessing in Vivo Efficacy of shRNA Expression Constructs Against HBV

To test the pG-U6shRNA series of plasmids against HBV in cell culture, Huh7 hepatoma cells were transfected with the target HBV construct, pCH-9/3091, together with a pG-U6shRNA construct, and the pLTR LacZ. pCH-9/3091 contains a terminally redundant genome of HBV subtype ayw, and expresses HBV antigens. pLTR LacZ constitutively produces β-galactosidase from a retroviral LTR promoter sequence and allows to control for transfection efficiency. In the positive control, pCH-9/3091 target plasmid was transfected together with pGEM-T Easy which lacked the short hairpin sequence. The negative control transfection did not contain pCH-9/3091 but included pCIneo, which does not contain HBV sequences. Lipofectamine was used as the transfecting agent, and the procedure was carried out according to the recommendations of the manufacturer (Invitrogen, CA, USA). Secretion of interferon alpha and beta by transfected hepatocytes was determined using a standard ELISA technique (R&D systems, MN, USA). None of the panel of shRNA plasmids was found to have an effect on the concentration of interferon alpha and beta in the culture supernatants (not shown). HBsAg secretion into the culture supernatants was measured daily using the Axsym (ELISA) immunoassay kits (Abbot Laboratories, IL, USA). FIG. 11A shows a representative sample of the effects of pG-U6shRNA plasmids on HBsAg secretion from Huh7 cells when cotransfected with pCH-9/3091. In a similar series of investigations, the pCH-EGFP construct was used as a source of target RNA transcripts. pCH-EGFP is derived from pCH-9/3091 [37], and contains a terminally redundant genome of HBV subtype ayw in which the preS2/S ORF was replaced with a sequence encoding the enhanced green fluorescent protein (EGFP) [38]. The efficacy of shRNA-encoding plasmids was assessed using flow cytometry to detect EGFP expression. The results from these investigations were similar to those obtained when using the intact HBV target expressing construct (pCH-9/3091) and are depicted in FIG. 11B. Northern blot hybridization was also performed according to standard procedures to determine the effects of shRNA-encoding plasmids on the concentration of HBV RNA within Huh7 cells. An HBV sequence from the surface region was radiolabelled using the multiprime technique (Megaprime kit, Amersham, UK) and then hybridized to resolved RNA that was extracted from transfected Huh7 cells. The RNA had been resolved using agarose gel electrophoresis and blotted onto nitrocellulose membranes prior to hybridization. These data are shown in the lower panel of FIG. 12. The cotransfected shRNA-encoding plasmids are indicated as well as the 3.5 kb and 2.4/2.1 kb HBV RNA transcripts that were isolated from the transfected cells. As a control for the blotting efficiency and equal loading of RNA samples, the same blot was stripped and reprobed using a radiolabelled GAPDH-specific probe. GAPDH is a constitutively active housekeeping gene that is expected to be present in similar concentrations in all of the cells transfected. The results from the northern blot analysis, measurements of HBV antigen secretion and flow cytometry to detect EGFP production from pCH-EGFP show that cotransfection of Huh7 cells with pCH-9/3091 and pG-U6shRNA10 or pCH-9/3091 and pG-U6shRNA11 substantially inhibit HBV gene expression in transfected cells in culture.

### EXAMPLE 4

### In Vivo Assessment of Efficacy of Anti HBV shRNA Constructs Using the Murine Hyperdynamic Tail Vein Injection Method

The murine hyperdynamic tail vein injection (MHI) method was employed to determine the effects of shRNA plasmid vectors on the expression of HBV genes in a small animal model of HBV infection. A large volume of DNA-containing saline solution is injected into the tail vein over a short period of time. Usually 10% of body mass (e.g. 2.8 ml of solution into a 28 g mouse) is injected over 5-10 seconds. This results in a rapid, but transient, rise in intrahepatic back pressure that delivers DNA efficiently to hepatocytes. Thus injection of pCH-9/3091 plasmid DNA results in expression that mimics HBV infection.

In a typical investigation, mice were injected with a combination of three plasmid sequences:

1 Target DNA: HBV-encoding plasmid DNA (pCH3091) or pCIneo plasmid DNA that lacks an insert (negative control)

2 Anti HBV sequence: shRNA-encoding plasmid DNA or backbone that lacks potentially therapeutic sequence

3 Control for hepatic DNA delivery: Constitutively active LTR LacZ-encoding plasmid (encoding β-galactosidase).

Representative examples of the effects of pG-U6shRNA10 on the expression of HBV antigens in mice injected with the pCH3091 plasmid together with LTR LacZ-encoding plasmid are represented in FIGS. 13 and 14. FIG. 13 shows the concentration of HBsAg in the serum of injected mice over a period of 4 days. Each curve represents the analysis from a single animal. Results from investigation of 4 control animals, which were injected with plasmid lacking the U6shRNA10 cassette are indicated. Four mice, which received pG-U6shRNA10 via tail vein injection are also shown. Compared to controls, it is clear that pG-U6shRNA10 decreases HBsAg expression to a level that is equivalent to the background. When liver sections from these mice were stained for β-galactosidase activity to control for the efficiency of DNA delivery, similar numbers, of transfected cells confirm equivalent and efficient expression of plasmids delivered via the tail veins (FIGS. 14 A&B). Detection of hepatic HBcAg using standard immunohistochemical procedures confirmed similar inhibition by pG-U6shRNA10 of the HBV antigen production in these liver sections (FIGS. 14 C&D) to that noted in culture studies. HBcAg was not detectable after co injection with pG-U6shRNA10 (FIG. 14D). FIG. 14 E is a representative high power field that shows specific immunohistochemical staining for HBcAg of the sample of FIG. 14 C. Taken together, these data demonstrate that pG-U6shRNA10 effects HBV gene knockdown such that HBV gene expression is substantially inhibited.

The sequences of the effective anti HBV shRNAs (shRNA 10 and shRNA 11), together with their HBV targets, are depicted in FIG. 15. The HBV Genome Sequence AY233287A is shown in SEQ. ID. NO. 13. Mismatches were incorporated into the stem region of the hairpins to facilitate generating the expression cassettes and also improve their processing to improve inhibition of target gene expression. The targeted sites within the entire HBV genome (Genbank sequence Number: AY233287A) are also indicated (SEQ. ID. NO. 13). For shRNA 10, the sequence is depicted in bold red font, and the target of shRNA11 is in italicised red font. The overlapping regions of the two targets are bold and in italics. Sequences shown in bold black font are those targeted by other shRNAs of the panel, which were found to be less effective inhibitors of markers of HBV gene expression than shRNA 10 and shRNA 11.

Combination of Hepatitis Delta Virus (HDV) Ribozyme and Hammerhead Ribozyme that Generates a shRNA Sequence

In FIG. 16, a schematic illustration is depicted that represents the use of a combination of hammerhead and HDV ribozymes to generate a shRNA sequence. Both ribozymes cleave in cis. Unlike with the combination of two hammerhead ribozymes, inclusion of a HDV ribozyme at the 3′ end allows its use as a universal cis cleaving ribozyme that could be used for generating any shRNA. Sequence requirements for HDV to be active are 3′ to the site of cleavage, and would be independent of the sequences incorporated into the hairpin.

The following references are incorporated herein by reference.
1. Wassenegger, M. and T. Pelissier, A model for RNA-mediated gene silencing in higher plants. Plant Mol Biol, 1998. 37(2): p. 349-62.
2. Ngo, H., et al., Double-stranded RNA induces mRNA degradation in Trypanosoma brucei. Proc Natl Acad Sci USA, 1998. 95(25): p. 14687-92.
3. Misquitta, L. and B. M. Paterson, Targeted disruption of gene function in Drosophila by RNA Interference (RNA-i): a role for nautilus in embryonic somatic muscle formation. Proc Natl Acad Sci USA, 1999. 96(4): p. 1451-6.
4. Caplen, N. J., et al., dsRNA-mediated gene silencing in cultured Drosophila cells: a tissue culture model for the analysis of RNA interference. Gene, 2000. 252(1-2): p. 95-105.
5. Li, Y. X., et al., Double-stranded RNA injection produces null phenotypes in zebrafish. Dev Biol, 2000. 217(2): p. 394-405.
6. Maine, E. M., A conserved mechanism for post-transcriptional gene silencing? Genome Biol, 2000. 1(3): p. 1018.
7. Hannon, G. J., RNA interference. Nature, 2002. 418(6894): p. 244-51.
8. Bernstein, E., et al., Role for a bidentate ribonuclease in the initiation step of RNA interference. Nature, 2001. 409(6818): p. 363-6.
9. Hamilton, A. J. and D. C. Baulcombe, A species of small antisense RNA in posttranscriptional gene silencing in plants. Science, 1999. 286(5441): p. 950-2.
10. Dalmay, T., et al., SDE3 encodes an RNA helicase required for post-transcriptional gene silencing in Arabidopsis. Embo J, 2001. 20(8): p. 2069-78.
11. Nykanen, A., B. Haley, and P. D. Zamore, ATP requirements and small interfering RNA structure in the RNA interference pathway. Cell, 2001. 107(3): p. 309-21.
12. Smardon, A., et al., EGO-1 is related to RNA-directed RNA polymerase and functions in germ-line development and RNA interference in C. elegans. Curr Biol, 2000. 10(4): p. 169-78.
13. Elbashir, S. M., W. Lendeckel, and T. Tuschl, RNA interference is mediated by 21- and 22-nucleotide RNAs. Genes Dev, 2001. 15(2): p. 188-200.
14. Fire, A., et al., Potent and specific genetic interference by double-stranded RNA in Caenorhabditis elegans. Nature, 1998. 391(6669): p. 806-11.
15. Kawasaki, H. and K. Taira, Induction of DNA methylation and gene silencing by short interfering RNAs in human cells. Nature, 2004.
16. Hutvagner, G. and P. D. Zamore, A microRNA in a multiple-turnover RNAi enzyme complex. Science, 2002. 297(5589): p. 2056-60.
17. Jiang, M. and J. Milner, Selective silencing of viral gene expression in HPV-positive human cervical carcinoma cells treated with siRNA, a primer of RNA interference. Oncogene, 2002. 21(39): p. 6041-8.
18. Zou, X., et al., Cdk4 disruption renders primary mouse cells resistant to oncogenic transformation, leading to Arf/p53-independent senescence. Genes Dev, 2002. 16(22): p. 2923-34.
19. Elbashir, S. M., et al., Duplexes of 21-nucleotide RNAs mediate RNA interference in cultured mammalian cells. Nature, 2001. 411(6836): p. 494-8.
20. Gil, J., M. Esteban, and D. Roth, In vivo regulation of the dsRNA-dependent protein kinase PKR by the cellular glycoprotein p67. Biochemistry, 2000. 39(51): p. 16016-25.
21. Player, M. R. and P. F. Torrence, The 2-5A system: modulation of viral and cellular processes through acceleration of RNA degradation. Pharmacol Ther, 1998. 78(2): p. 55-113.
22. Kumar, M. and G. G. Carmichael, Antisense RNA: function and fate of duplex RNA in cells of higher eukaryotes. Microbiol Mol Biol Rev, 1998. 62(4): p. 1415-34.
23. Agrawal, S. and Q. Zhao, Antisense therapeutics. Curr Opin Chem Biol, 1998. 2(4): p. 519-28.
24. Miyagishi, M. and K. Taira, U6 promoter-driven siRNAs with four uridine 3′ overhangs efficiently suppress targeted gene expression in mammalian cells. Nat Biotechnol, 2002. 20(5): p. 497-500.
25. Lee, N. S., et al., Expression of small interfering RNAs targeted against HIV-1 rev transcripts in human cells. Nat Biotechnol, 2002. 20(5): p. 500-5.
26. Brummelkamp, T. R., R. Bernards, and R. Agami, A system for stable expression of short interfering RNAs in mammalian cells. Science, 2002. 296(5567): p. 550-3.
27. Paddison, P. J., et al., Short hairpin RNAs (shRNAs) induce sequence-specific silencing in mammalian cells. Genes Dev, 2002. 16(8): p. 948-58.
28. Sui, G., et al., A DNA vector-based RNAi technology to suppress gene expression in mammalian cells. Proc Natl Acad Sci USA, 2002. 99(8): p. 5515-20.
29. Yu, J. Y., S. L. DeRuiter, and D. L. Turner, RNA interference by expression of short-interfering RNAs and hairpin RNAs in mammalian cells. Proc Natl Acad Sci USA, 2002. 99(9): p. 6047-52.
30. Paul, C. P., et al., Effective expression of small interfering RNA in human cells. Nat Biotechnol, 2002. 20(5): p. 505-8.
31. McManus, M. T., et al., Gene silencing using micro-RNA designed hairpins. Rna, 2002. 8(6): p. 842-50.
32. Grishok, A., et al., Genes and mechanisms related to RNA interference regulate expression of the small temporal RNAs that control C. elegans developmental timing. Cell, 2001. 106(1): p. 23-34.
33. Kawasaki, H. and K. Taira, Short hairpin type of dsRNAs that are controlled by tRNA(Val) promoter significantly induce RNAi-mediated gene silencing in the cytoplasm of human cells. Nucleic Acids Res, 2003. 31(2): p. 700-7.
34. Xia, H., et al., siRNA-mediated gene silencing in vitro and in vivo. Nat Biotechnol, 2002. 20(10): p. 1006-10.

SEQ ID NO.1

5′-CTAGACAGCCCTGATGAGTCCGTGAGGACGAAACTTGATCAAAGTCG
GCTGTAGGC-3′,

SEQ ID NO.2

5′-CAATTTATGCCTACAGCCGACTTTGATCAAGTTTCGTCCTCACGGAC
TCATCAGGGCTGT-3′,

SEQ ID NO.3

5′-ATAAATTGGTTTGCGAGGTGCGCACTGATGAGTCCGTGAGGACGAAA
CCAATTACTAGTG-3′,

SEQ ID NO.4

5′-AATTCACTAGTAATTGGTTTCGTCCTCACGGACTCATCAGTGCGCAC
CTCGCAAAC-3′

SEQ ID NO.5

5′-CTAGAATTGGCTGATGAGTCCGTGAGGACGAAACTTGATCAAAGTCC
CAATTTATG-3′,

SEQ ID NO.6

5′-GCTGTAGGCATAAATTGGGACTTTGATCAAGTTTCGTCCTCACGGAC
TCATCAGCCAATT-3′,

SEQ ID NO.7

5′-CCTACAGCCTTAGTAGGTGACCTCTGATGAGTCCGTGAGGACGAAAG
GCTGTACTAGTG-3′,

SEQ ID NO.8

5′-AATTCACTAGTACAGCCTTTCGTCCTCACGGACTCATCAGAGGTCAC
CTACCTAAG-3′
Ribozyme-shRNA1
(targeting HBV coordinates 1514-1538)

SEQ ID NO. 9

5′-GATCCTCGAGTCTAGACGCCTGATGAGTCCGTGAGGACGAAACGAAT
TCTGATCAGAATTCGTC GCGCGACCACGGGGCGCACCTCTCTTTACTGT
GAAGCCACAGATGGGCAAAGAGAGATGCGCCCCATGGCCGTGCTC GGGC
CCCCGCGGGGGCCCCTGATGAGAGGAGTG CCTCGAAAGCAACTAGTGCG
GCCGCTAAACTAT-3,

SEQ ID NO. 10

5′-GATCCTCGAGTCTAGACGCCTGATGAGTCCGTGAGGACGAAACGAAT
TCTGATCAGAATTCGTC GCGCCGTGTGCACTTCGCTTCACCTCTGCTGT
GAAGCCACAGATGGGCAGAGGCGAAGCAAAGCGCACACGATGCTC GGGC
CCCCGCGGGGGCCCCTGATGAGAGGAGTGCCTCGAAAGCAACTAGTGCGG
CCGCTAAACTAT-3′
Ribozyme-shRNA3
(targeting HBV coordinates 1863-1887)

SEQ ID NO. 11

5′-GATCCTCGAGTCTAGACGCCTGATGAGTCCGTGAGGACGAAACGAAT
TCTGATCAGAATTCGTC GCGTTCAAGCCTCCAAGCTGTGCCTTGGCTGT
GAAGCCACAGATGGGCCAAAGCACAACTCGGAGGCTCGAATGCTC GGGC
CCCCGCGGGGGCCCCTGATGAGAGGAGTGCCTCGAAAGCAACTAGTGCGG
CCGCTAAACTAT-3′

SEQ ID NO. 12

5′-CTAGACAGCCCTGATGAGTCCGTGAGGACGAAACTTGATCAAAGTCG
GCTGTAGGCATAAATTGGTTTGCGAGGTGCGCACTGATGAGTCCGTGAGG
ACGAAACCAATTACTAGTGCTAGAATTGGCTGATGAGTCCGTGAGGACGA
AACTTGATCAAAGTCCCAATTTATGCCTACAGCCTTAGTAGGTGACCTCT
GATGAGTCCGTGAGGACGAAAGGCTGTACTAGTG-3′

SEQ ID 13

5′-TTCCACAGCTTTCCACCAAGCTCTGCAAGATCCCAGAGTCAGGGGCC
TGTATTTTCCTGCTGGTGGCTCCAGTTCAGGAACACTCAACCCTGTTCCA
AATATTGCATCTCACATCTCGTCAATCTCCTCGAGGATTGGGGACCCTGC
GCTGAACATGGAGAACATTACATCGGATTCCTAGGACCCCTGCTCGTGTT
ACAGGCGGGGTTTTTCTTGTTGACAAGAATCCTCACAATACCGCAGAGTC
TAGACTCGTGGTGGACTTCTCTCAATTTTCTAGGGGGATCACCCGTGTGT
CTTGGCCAAAATTCGCAGTCCCCAACCTCCAATCACTCACCAACCTCCCG
TCCTCCAATTTGTCCTGGTTATCGCGGGATGTGTCTGCGGCGTTTTATCA
TATTCCTCTTCATCCTGCTGCTATGCCTCATCTTCTTATTGGTTCTTCTG
GATTACCAAGGTATGTTGCCCGTTTGTCCTCTAATTCCAGGATCAACAAC
AACCAGTACGGGACCCTGCAAAACCTGCACGACTCCTGCTCAAGGCAACT
CTATGTTTCCCTCATGTTGCTGTACAAAACCTACGGATGGAAATTGCACC
TGTATTCCCATCCCATCATCTTGGGCTTTCGCAAAATACCTATGGGAGTG
GGTCTCAGTCCGTTTCTCTTGGCTCAGTTTACTAGTGCCATTTGTTCAGT
GGTTCGTAGGGCTTTCCCCCACTGCTTGGCTTTCAGCTATATGGATGATG
TGGTATTGGGGGCCAAGTCTGTACAACATCTTGAGTCCCTTTATACCGCT
GTTACCAATTTTCTTTTGTCTTTGGGTATACATTTAAACCCTAACAAAAC
AAAGAGATGGGGTTATTCCCTAAACTTCATGGGATATGTAATTGGAAGTT
GGGGTACGTTGCCACAGGATCATATTGTACAAAAAATCAAATGCTGTTTT
AGAAAACTTCCTGTCAATCGACCTATTGATTGGAAAGTATGTCAAAGAAT
TGTGGGTCTTTTGGGCTTTGCCGCTCCATTTACACAATGTGGTTACCCTG
CCTTAATGCCTTTGTATGCATGTATACAAGCAAAACAGGCTTTTACTTTC
TCGCCAACTTACAAGGCCTTTCTAAGTCAACAGTATATGAACCTTTACCC
CGTTGCCCGGCAACGGCCTGGTCTGTGCCAAGTGTTTGCTGAGCAACCCC
CACTGGCTGGGGCTTGGCTATCGGCCATCAGCGCATGAGTGGAACCTTTG
TGGCTCCTCTGCCGATCCATACTGCGGAACTCCTAGCTGCTTGTTTTGCT
CGCAGCAGGTCTGGAGCAAAACTCATCGGGACTGATAATTCTGTCGTCCT
TTCTCGGAAATATACATCATTTCCATGGCTGCTAGGTTGTACTGCCAACT
GGATTCTTCGCGGGACGTCCTTTGTTTACGTCCCGTCGGCGCTGAATCCC
GCGGACGACCCCTCGCGGGGCCGCTTGGGACTCTATCGTCCCCTTCTCCG
TCTGCCGTACCGTCCGACCACGGGGCGCACCTCTCTTTACGCGGTCTCCC
CGTCTGTGCCTTCTCATCTGCCGGTCCGTGTGCACTTCGCTTCACCTCTG
CACGTTGCATGGAGACCACCGTGAACGCCCATCAGATCCTGCCCAAGGTC
TTACATAAGAGGACTCTTGGACTCCCAGCAAATGTCAAGCGACCGACCTT
GAGGCCTACTTCAAAGACTGTGTGTTTAAAGACTGGGAGGAGTTGGGGGA
GGAGATTAGGTTAAAGGTCTTTGTATTAGGAGGCTGTAGGCATAAATTGG
TCTGCGCACCATCATCATGCAACTTTTTCACCTCTGCCTAATCATCACTT
GTACATGTCCCACTCTTCAAGCCTCCAAGCTGTGCCTTGGATGGCTTTGG
GACATGGACATTGACCCTTATAAAGAATTTGGAGCTACTGTGGAGTTACT
CTCATTTTTGCCTTCTGACTTCTTTCCTTCAGTCCGGGATCTACTTGATA
CAGCTTCAGCTCTGTATCGGGAAGCCTTAGAGTCTCCGGAGCATTGCTCC
CCTCACCATACAGCACTCAGGCAAGCCATTCTCTGCTGGGGGGAATTAAT
GGCTCTAGCCACCTGGGTGGGTAATAATTTGGAAGATCCAGCATCCAGGG
ATCTAGTAGTCAATTATGTTAACACTAACATGGGCCTAAAGATCAGACAA
CTATTGTGGTTTCATATTTCTTGCCTTACTTTTGGAAGAGAAACTGTCCT
TGAGTATTTGGTCTCTTTCGGAGTGTGGATTCGCACTCCTCCAGCCTATA
GACCACCAAATGCCCCTATCCTATCAACACTTCCGGAAACTACTGTTGTT
AGACGACGAGATCGAGGCAGGTCCCCTAGAAGAAGAACTCCCTCGCCTCG
CAGACGAAGATCTCAATCGCCGCGTCGCAGAAGATCTCAATCTCGGGAAT
CTCAATGTTAGTATTCCTTGGACTCATAAGGGGGGAAACTTTACTGGGCT
TTATTCCTCTACTGTCCCTATCTTTAATCCTGAATGGCAAACTCCTTCTT
TTCCTAAAATTCATTTACATGAGGACATTATTAATAGGTGTCAGCAATTT
GTAGGCCCTTTAACTGTAAATGAAAAGAGAAGATTAAAATTAATTATGCC
TGCTAGATTTTATCCAAACAGCACCAAATATTTGCCTCTAGACAAAGGGA
TTAAGCCTTATTATCCTGATCAAGTAGTTAATCATTACTTCCAGACCAGA
CATTATTTACATACTCTTTGGAAGGCTGGGATTCTATATAAGAGGGAAAC
TACACGTAGCGCCTCATTTTGCGGGTCACCATATTCTTGGGAACAAGAGC
TACATCATGGGAGGTTGGTCAACAAAACCTCGCAAAGGCATGGGGACGAA
TCTTTCTGTTCCCAACCCTCTGGGATTCTTTCCCGATCATCAGTTGGACC
CTGCATTCGGAGCCAATTCAAACAATCCAGATTGGGACTTCAACCCCATC
AAGGACCACTGGCCACAAGCCAACCAGGTAGGAGTGGGAGCATTCGGGCC
AGGGTTCACTCCCCCACACGGAGGTGTTTTGGGGTGGAGCCCTCAGGCTC
AGGGCATATTGGCTACAGTGCCAACAGTTCCTCCTCCTGCCTCCACCAAT
CGGCAGTCAGGAAGGCAGCCTACTCCCATCTCTCCACCTCTAAGAGACAG
TCATCCTCAGGCCATGCAGTGGAA-3′

SEQ ID 14

5′. . . GUCGUGUGCGCUUUGCUUCGCCUCUGCUUCCUGUCACAGAGG
UGAAGCGAAGUGCACACGG . . . 3′

SEQ ID 15

5′. . . GUACACCUCGCUUCACCUCUACACAUCUUCCUGUCAACGUGC
AGAGGUGAAGCGAAGUGGA . . . 3′

1. A self-processing RNA expression cassette which includes
at least one pair of processing units,
an RNAi effecter sequence of predetermined length that regulates target gene expression which is flanked by said pair of processing units; wherein the RNAi effecter sequence includes at least one target recognition sequence derived from Hepatitis B Virus (HBV); and
at least one pair of cognate ribozyme cis-cleavage target sites located 5′ and 3′ of the RNAi effecter sequence.

2. A self-processing RNA expression cassette according to claim 1, wherein the expression cassette is able to express in vivo and in vitro.

3. A self-processing RNA expression cassette according to claim 1, wherein the expression cassette is able to express in vitro by means of a bacteriophage promoter.

4. A self-processing RNA expression cassette according to claim 1, wherein the processing units are a pair of ribozymes which include
a first-ribozyme, or part thereof, having a first cis-cleavage specificity, the first-ribozyme or part thereof having cis-cleavage activity and including a catalytic domain and an antisense domain;
a second-ribozyme or part thereof having a second cis-cleavage specificity, the ribozyme or part thereof having cis-cleavage activity and including a catalytic domain and an antisense domain.

5. A self-processing RNA expression cassette according to claim 1, wherein the RNAi effecter sequence of predetermined length includes at least one target recognition sequence derived from the Hepatitis B Virus (HBV) X gene (HBx).

6. A self-processing RNA expression cassette according to claim 1, wherein the self-processing RNA expression cassette is multimeric.

7. A nucleic acid sequence which is selected from the group consisting of SEQ ID NO. 9; a nucleic acid sequence complementary to SEQ ID NO. 9; a nucleic acid sequence which hybridizes specifically to SEQ ID NO. 9; a sequence of a hepadnavirus homologous to SEQ ID NO. 9; and a nucleic acid sequence which has at least 90% sequence identity to one of said sequences.

8. A nucleic acid sequence which is selected from the group consisting of SEQ ID NO. 10; a nucleic acid sequence complementary to SEQ ID NO. 10; a nucleic acid sequence which hybridizes specifically to SEQ ID NO. 10; a sequence of a hepadnavirus homologous to SEQ ID NO. 10; and a nucleic acid sequence which has at least 90% sequence identity to one of said sequences.

9. A nucleic acid sequence which is selected from the group consisting of SEQ ID NO. 11; a nucleic acid sequence complementary to SEQ ID NO. 11; a nucleic acid sequence which hybridizes specifically to SEQ ID NO. 11; a sequence of a hepadnavirus homologous to SEQ ID NO. 11; and a nucleic acid sequence which has at least 90% sequence identity to one of said sequences.

10. A nucleic acid sequence which is selected from the group consisting of SEQ ID NO. 12; a nucleic acid sequence complementary to SEQ ID NO. 12; a nucleic acid sequence which hybridizes specifically to SEQ ID NO. 12; a sequence of a hepadnavirus homologous to SEQ ID NO. 12; and a nucleic acid sequence which has at least 90% sequence identity to one of said sequences.

11. A nucleic acid sequence which is selected from the group consisting of SEQ ID NO. 14; a nucleic acid sequence complementary to SEQ ID NO. 14; a nucleic acid sequence which hybridizes specifically to SEQ ID NO. 14; a homologous sequence of a hepadnavirus; or a nucleic acid sequence which has at least 90% sequence identity to one of said sequences.

12. A nucleic acid sequence which is selected from the group consisting of SEQ ID NO. 15; a nucleic acid sequence complementary to SEQ ID NO. 15; a nucleic acid sequence which hybridizes specifically to SEQ ID NO. 15; a homologous sequence of a hepadnavirus; or a nucleic acid sequence which has at least 90% sequence identity to one of said sequences.

13. A nucleic acid sequence according to claim 11 which further includes the sequence CCGTGTGCACTTCGCTTCACCTCTG or part thereof; a complementary nucleic acid sequence or part thereof; or a nucleic acid sequence which has at least 90% sequence identity to one of said sequences.

14. A nucleic acid sequence according to claim 12 which further includes the sequence TGCACTTCGCTTCACCTCTGCACGT or part thereof; a complementary nucleic acid sequence or part thereof; or a nucleic acid sequence which has at least 90% sequence identity to one of said sequences.

15. A method of inhibiting expression of at least one target DNA sequence having at least one target recognition sequence, the method including the steps of:
providing a nucleic acid sequence encoding an expression construct having a self-processing RNA expression cassette according to claim 1, wherein cis-cleavage activity domains of the ribozymes recognise specific cleavage sites within the self-processing RNA expression cassette, said cis-cleavage sites being arranged within the self-processing RNA expression cassette in such a manner that the cis-cleavage activity of said ribozymes produces an RNAi effecter sequence of predetermined length that regulates target gene expression;
expressing the nucleic acid sequence encoding the self-processing RNA expression cassette to produce the self-processing RNA expression cassette;
producing an RNAi effecter sequence of pre-determined length, or precursor thereof, that regulates target gene expression after cis-cleavage of RNA produced from the expression cassette; and
allowing the cleaved RNAi effecter sequence, to contact at least one target DNA sequence, whereby the said effecter sequence, directs the inhibition of expression of the target DNA.

16. A method of inhibiting expression of at least one target RNA transcript having at least one target recognition sequence, the method including the steps of:
providing a nucleic acid sequence encoding an expression construct having a self-processing RNA expression cassette according to claim 1, wherein cis-cleavage activity domains of the ribozymes recognise specific cleavage sites within the self-processing RNA expression cassette, said cis-cleavage sites being arranged within the self-processing RNA expression cassette in such a manner that the cis-cleavage activity of said ribozymes produces an RNAi effecter sequence of a pre-determined length;
expressing the nucleic acid sequence encoding the self-processing RNA expression cassette to produce the self-processing RNA expression cassette;
producing a RNAi effecter molecule, or precursor thereof, of pre-determined length by cis-cleavage of RNA produced from the expression cassette; and
allowing the cleaved RNAi effecter molecule, to contact at least one target RNA transcript, whereby the RNAi effecter molecule, directs the inhibition of expression of the target RNA transcript(s).

17. A method of producing at least one RNAi effecter sequence, the method including the steps of:
providing a nucleic acid sequence encoding an expression construct having a self-processing RNA expression cassette which includes
at least one pair of processing units,
an RNAi effecter sequence of predetermined length that regulates target gene expression which is flanked by said pair of processing units; and
at least one pair of cognate ribozyme cis-cleavage target sites located 5′ and 3′ of the RNAi effecter sequence, wherein cis-cleavage activity domains of the ribozymes recognise specific cleavage sites within the self-processing RNA expression cassette, said cis-cleavage sites being arranged within the self-processing RNA expression cassette in such a manner that the cis-cleavage activity of said ribozymes produces an RNAi effecter sequence of predetermined length that regulates target gene expression;
expressing the nucleic acid sequence encoding the self-processing RNA expression cassette in vitro to produce the self-processing RNA expression cassette; and
producing an RNAi effecter sequence of pre-determined length, or precursor thereof, that regulates target gene expression after cis-cleavage of RNA produced from the expression cassette.

18. A method of producing at least one RNAi effecter sequence, the method including the steps of:
providing a nucleic acid sequence encoding an expression construct having a self-processing RNA expression cassette which includes
at least one pair of processing units,
an RNAi effecter sequence of predetermined length that regulates target gene expression which is flanked by said pair of processing units; and
at least one pair of cognate ribozyme cis-cleavage target sites located 5′ and 3′ of the RNAi effecter sequence, wherein cis-cleavage activity domains of the ribozymes recognise specific cleavage sites within the self-processing RNA expression cassette, said cis-cleavage sites being arranged within the self-processing RNA expression cassette in such a manner that the cis-cleavage activity of said ribozymes produces an RNAi effecter sequence of predetermined length that regulates target gene expression;
expressing the nucleic acid sequence encoding the self-processing RNA expression cassette in silico to produce the self-processing RNA expression cassette; and
producing an RNAi effecter sequence of pre-determined length, or precursor thereof, that regulates target gene expression after cis-cleavage of RNA produced from the expression cassette.

19. A method of producing at least one RNAi effecter sequence according to claim 17, wherein the step of expressing the nucleic acid sequence encoding the self-processing RNA expression cassette is performed using a bacteriophage promoter.

20. A method of producing at least one RNAi effecter sequence according to claim 18, wherein the step of expressing the nucleic acid sequence encoding the self-processing RNA expression cassette is performed using a bacteriophage promoter.

21. A method of producing at least one RNAi effecter sequence according to claim 19, wherein the method includes the further step of purification of the RNAi effecter sequence.

22. (canceled)

23. A vector having incorporated therein a nucleic acid sequence according to claim 7.

24. (canceled)

25. A method of regulating the expression of DNA, the method including the steps of:
generating in silico a self-processing RNA expression cassette sequence according to claim 1 to produce an RNAi effecter sequence of predetermined length that regulates target gene expression by:
self-processing of the RNA expression cassette into its individual processing units and a RNA sequence that comprises an RNAi effecter;
separating or at least partially purifying the RNAi effecter; and
introducing the separated RNAi effecter sequence into a cell whereupon it is processed intracellularly to act on a target RNA transcript, thereby inhibiting the expression of the target sequence or subsequence thereof.

26. A method of producing at least one RNAi effecter sequence according to claim 20, wherein the method includes the further step of purification of the RNAi effecter sequence.

27. A vector having incorporated therein a nucleic acid sequence according to claim 8.

28. A vector having incorporated therein a nucleic acid sequence according to claim 9.

29. A vector having incorporated therein a nucleic acid sequence according to claim 10.

30. A vector having incorporated therein a nucleic acid sequence according to claim 11.

31. A vector having incorporated therein a nucleic acid sequence according to claim 12.

32. A vector having incorporated therein a nucleic acid sequence according to claim 13.

33. A vector having incorporated therein a nucleic acid sequence according to claim 14.

34. A composition which includes a vector according to claim 23 and a physiologically acceptable carrier.

35. A composition which includes a vector according to claim 27 and a physiologically acceptable carrier.

36. A composition which includes a vector according to claim 28 and a physiologically acceptable carrier.

37. A composition which includes a vector according to claim 29 and a physiologically acceptable carrier.

38. A composition which includes a vector according to claim 30 and a physiologically acceptable carrier.

39. A composition which includes a vector according to claim 31 and a physiologically acceptable carrier.

40. A composition which includes a vector according to claim 32 and a physiologically acceptable carrier.

41. A composition which includes a vector according to claim 33 and a physiologically acceptable carrier.

42. A cell having incorporated therein a nucleic acid sequence according to claim 7.

43. A cell having incorporated therein a nucleic acid sequence according to claim 8.

44. A cell having incorporated therein a nucleic acid sequence according to claim 9.

45. A cell having incorporated therein a nucleic acid sequence according to claim 10.

46. A cell having incorporated therein a nucleic acid sequence according to claim 11.

47. A cell having incorporated therein a nucleic acid sequence according to claim 12.

48. A cell having incorporated therein a nucleic acid sequence according to claim 13.

49. A cell having incorporated therein a nucleic acid sequence according to claim 14.

50. Use of a self-processing RNA expression cassette according to claim 1, in the manufacture of a preparation for treating Hepatitis B Virus (HBV) infection, or diseases caused thereby.
